(19) United States
(12) Patent Application Publication
(10) Pub. No.: US 2010/0040558 A1
(43) Pub. Date: Feb. 18, 2010

(54) GREEN TEA POLYPHENOL ALPHA SECRETASE ENHANCERS AND METHODS OF USE

(75) Inventors: R. Douglas Shytle, Largo, FL (US); Jun Tan, Tampa, FL (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: UNIVERSITY OF SOUTH FLORIDA, Tampa, FL (US)

(21) Appl. No.: 11/919,444

(22) PCT Filed: Apr. 26, 2006
(86) PCT No.: PCT/US2006/015884

§ 371 Date: Nov. 2, 2009

(51) Int. Cl. A61K 9/12; C12Q 1/37; C12N 5/02; A61K 31/35; A61K 31/05

(52) U.S. Cl. 424/45; 435/23; 435/375; 514/456; 514/734

(57) ABSTRACT

The subject invention concerns materials and methods for treating or preventing a neurodegenerative condition or disease associated with β-amyloid peptide deposition in neural tissue in a person or animal by administering a therapeutically effective amount of a polyphenol, or an analog, isomer, metabolite, or prodrug thereof, that increases expression or activity of a protein that exhibits α-secretase activity. The subject invention also provides methods to increase α-secretase expression and/or activity in cells by administering polyphenol flavonoids like (−)-epigallocatechin-3-gallate (EGCG) and epicatechin (EC), two polyphenols derived from green tea and other plants and that can be produced synthetically. Furthermore, there are provided methods to decrease or inhibit the production of Aβ₁₋₄₀ or Aβ₁₋₄₂ by administering the EGCG and EC compounds, their analogs, metabolites, and prodrugs.

### CROSS-REFERENCE TO A RELATED APPLICATION

This application claims the benefit of U.S. Provisional Application Ser. No. 60/675,060, filed Apr. 26, 2005, which is hereby incorporated by reference herein in its entirety, including any figures, tables, and drawings.

### BACKGROUND OF THE INVENTION

Green tea, the beverage made from the unfermented leaves of camellia sinensis, is one of the most ancient and widely consumed beverages in the world. Green tea polyphenols have demonstrated significant antioxidant properties. On the basis of a large body of evidence, it has become clear that compounds from green tea play different roles in antioxidant and other functions.

There is increasing evidence supporting the central role of antioxidant effects in opposing aging-related diseases. Recent studies suggest that green tea may be employed for the prevention and treatment of multiple neurodegenerative diseases including AD and other forms of dementia (Okello et al., 2004). However, there are no reports as to the active ingredients in green tea that have beneficial effects on neurodegenerative conditions such as Alzheimer's disease.

Amyloid precursor protein (APP) proteolysis is the fundamental process for the production of β-amyloid (Aβ) peptides which can be deposited as plaques in brain tissue and which are implicated in Alzheimer's disease (AD) pathology (Golde et al., 2000; Huse and Doms, 2000; Sambamurti et al., 2002; Funamoto et al., 2004). APP proteolytic products arise from the coordinated action of α-, β, and γ-secretases. In the amyloidogenic pathway, Aβ peptides are produced by the initial action of β-secretase (FACE) cleavage, which creates an Aβ-containing C-terminal fragment (CTF) known as β-CTF or C99 (Sinha and Lieberburg, 1999; Yan et al., 1999). This proteolysis also generates an N-terminal, soluble APP-β (sAPP-β) fragment, which is released extracellularly. Intracellularly, β-CTF is then cleaved by a multi-protein γ-secretase complex that results in generation of the Aβ peptide and a smaller γ-CTF, also known as C57 (De Strooper et al., 1998; Steiner et al., 1999). Conversely, in the nonamyloidogenic pathway, APP is first cleaved at the α-secretase site, which results in the release of N-terminal sAPP-α and the generation of α-CTF or C83 (Hooper and Turner, 2002), events that are indicative of a α-secretase activity (Hooper and Turner, 2002). Cleavage within the Aβ domain of APP results in two nonamyloidogenic pieces and thereby prevents Aβ peptide generation from that APP (Lichtenthaler et al., 2004). Because of the limiting amount of APP in the cell and the failure to saturate the BACE pathway during APP overexpression, it is believed that the above-mentioned amyloidogenic and nonamyloidogenic pathways compete for substrate in the process of APP proteolysis (Gandhi et al., 2004). Therefore, it is often inferred that extracellular elevation of sAPP-α (resulting from nonamyloidogenic pathway activation) can be taken as indirect evidence of inhibition of BACE and the associated amyloidogenic pathway. However, because the extracellular secretion of these various fragments can be regulated independently of APP cleavage, it is important to fully characterize the effects of treatment on both pathways concurrently before making inferences about underlying mechanisms (Rossner et al., 2000).

Over the past decade, intense focus has been given to investigating the processes of APP proteolysis and Aβ metabolism as possible targets for AD therapy (Hardy and Selkoe, 2002). Various synthetic and naturally occurring compounds have been analyzed for their efficacy in the modulation of these pathological events. One such naturally occurring compound achieving worldwide popularity for its therapeutic application is green tea. Green tea contains polyphenolic structures categorized as flavonoids, which are believed to be the active components accounting for the therapeutic properties of green tea. One green tea compound, (−)-epigallocatechin-3-gallate (EGCG), has been extensively studied primarily because of its reported anticarcinogenic effects (Lin and Liang, 2000; Moyers and Kumar, 2004). Recently, EGCG has been found to modulate protein kinase C (PKC) activity and to consequently increase secreted levels of sAPP-α (Levites et al., 2002; Levites et al., 2003). Additionally, EGCG has been shown to inhibit various activities of proinflammatory cytokines (Ahmed et al., 2002; Han, 2003; Li et al., 2004). Accordingly, signal transducer and activator of transcription 1 and nuclear factor κB responses are inhibited by EGCG (Han, 2003; Aktas et al., 2004). Elucidation of these molecular actions of EGCG substantiates the compound as a versatile modulator of cellular responses that may contribute to disease pathogenesis.

A number of reports have implicated members of the α-disintegrin-and-metalloprotease (ADAM) family, a family of zinc metalloproteases that includes ADAM9, 10, and 17, as putative α-secretase candidates (Hooper et al., 2002; Allinson et al, 2003; Asai et al., 2003). Lammich and colleagues first described the ability of ADAM10 to act as an α-secretase (Lammich et al., 1999), whereas Buxbaum and co-workers reported that ADAM17 contributes to α-secretase processing of APP (Buxbaum et al., 1998). Others have demonstrated the ability of ADAM9 to promote α-secretase cleavage (Hotoda et al., 2002). However, Asai and colleagues reported that ADAM9, 10, and 17 all have roles in the processing of APP to sAPP-α in vitro (Asai et al., 2003). In cerebrospinal fluid from AD patients, ADAM10 and corresponding sAPP/α-CTFs are decreased (Colciaghi et al., 2002; Colciaghi et al., 2004). Moreover, ADAM10 is also decreased in AD and Down's syndrome brains (Bernstein et al., 2003). A report by Lopez-Perez and colleagues implicates ADAM10 as a contributor to constitutive sAPP-α production, whereas ADAM17 (also known as TNF-α converting enzyme, TACE) is implicated in a regulated mechanism of sAPP-α production (Lopez-Perez et al., 2001). Recently, Postina et al., 2004 showed that activation of α-secretase significantly reduces AD-like pathology in an animal model of AD (Postina et al., 2004).

### BRIEF SUMMARY OF THE INVENTION

The subject invention concerns materials and methods for treating or preventing a neurodegenerative condition or disease associated with β-amyloid peptide deposition in neural tissue in a person or animal by administering a therapeutically effective amount of a polyphenol, or an analog, isomer, metabolite, or prodrug thereof, that increases expression or activity of a protein that exhibits α-secretase activity. In one embodiment, the protein that exhibits α-secretase activity is ADAM10. Polyphenols contemplated within the scope of the methods of the invention include epigallocatechin-3-gallate (EGCG) and epicatechin (EC). In one embodiment, the neurodegenerative disease or condition to be treated is Alzheimer's disease. In one embodiment, the polyphenol increases the cleavage activity of the protein having α-secretase activity. In another embodiment, the polyphenol increases the expression of the gene encoding the protein and/or increases the amount of the protein produced or present in a cell.

The subject invention provides methods to increase α-secretase expression and/or activity in cells by administering polyphenol flavonoids like (−)-epigallocatechin-3-gallate (EGCG) and epicatechin (EC), two polyphenols derived from green tea and other plants and that can be produced synthetically. Furthermore, there are provided methods to decrease or inhibit the production of Aβ₁₋₄₀ or Aβ₁₋₄₂ by administering the EGCG and EC compounds, their analogs, metabolites, and prodrugs. Treatment of certain mammalian cells with certain green tea derived polyphenols have shown that the polyphenols decrease Aβ₁₋₄₂ and Aβ₁₋₄₀ peptide in a dose dependent manner.

In one embodiment, there are provided methods for treating an amyloid disease in a mammalian patient, comprising administering to the patient an effective amount of a polyphenol that increases expression and/or activity of a protein having α-secretase functional activity.

In another embodiment, there are provided methods for treating elevated levels of amyloid peptides in cells or in a mammalian patient. Specifically, the subject methods can be used to inhibit and/or reduce β-amyloid peptide (Aβ production within a cell.

In yet another embodiment, there are provided methods for enhancing the cleavage of tumor necrosis factor α-converting enzyme (TACE). Without being limited by theory, this upregulation of TACE likely promotes α-secretase cleavage of APP.

In another embodiment, there are provided methods for decreasing the expression and/or cleavage activity of α-secretase in a mammalian cell by administering polyphenols, specifically, gallic acid monohydrate, catechin, and catechin hydrate.

### BRIEF DESCRIPTION OF DRAWINGS

FIGS. 1A-1D show EGCG treatment inhibits Aβ generation in cultured neuronal cells. Aβ₁₋₄₀ and ₁₋₄₂ peptides were analyzed in the conditioned media from SweAPP N2a cells (FIGS. 1A, 1C, 1D) and transgenic APP_{sw} mouse-derived primary neuronal cells (FIG. 1B) by ELISA (n=3 for each condition). Data are represented as percentage of Aβ secreted 12 hours after EGCG treatment relative to control (PBS). For FIGS. 1A, 1B, a t-test revealed significant between EGCG- and either of other compounds-treated condition at 40, 20, 10 and 5 μM (P<0.001). For FIG. 1C, in comparison of EGCG (20 μM)- and co-treated SweAPP N2a cells with EGCG (20 μM) plus GC (80 μM), Catechin (C) (80 μM) or GC/C, there is a significant difference between EGCG treatment and either of GC, C and GC/C (P<0.001). For FIG. 1D, given that SweAPP N2a cells were treated with EGCG at a relative same concentration (green tea extract (GI) contains 30% EGCG), there is a significant difference between GT and EGCG treatments (40 μg/mL versus 20 μM; 20 μg/mL versus 10 μM; 10 μg/mL versus Aβ in inhibition of Aβ generation (P<0.001). Reduction for each treatment and various combinations is indicated for (c, d).

FIGS. 2A-2H show EGCG treatment alters APP cleavage processing in vitro. (FIGS. 2A, 2B) SweAPP N2a cells were treated with EGCG at 20 μM or PBS (control) for 12 hours. Cell cultured supernatants were collected and cell lysates were prepared from these cultured cells. (FIGS. 2C, 2D) Cell lysates were prepared from SweAPP N2a cells treated with EGCG at 20 μM for a wild range of time points (c) or EGCG at various doses for 12 hours (FIG. 2D). (FIGS. 2E, 2F, 2G, 2H) Cell lysates were prepared from SweAPP N2a cells treated with EGCG (−) EC, (+) EC, GC, C, GT or various combinations at the different dose as indicated for 12 hours. Western blot analysis by antibody 369 against the cytoplasmic tail of APP shows holo APP, and two bands corresponding β-CTF (C99) and α-CTF (C83). For (FIG. 2B), Western blot analysis by antibody 22C11 against the N-terminal of APP shows sAPP-α [immunoprecipitation (IP) with 6E10 (against Aβ1-17)] and sAPP-β (after IP with 6E10, IP again with 22C11). For FIGS. 2C, 2D, Western blot by anti-Actin antibody shows Actin protein (as an internal referent control). We observed the similar data described above in N2a cells transfected with human wild-type APP695 following EGCG treatment.

FIGS. 3A-3E show EGCG treatment promotes α-secretase cleavage of APP in vitro. (FIGS. 3A, 3B) Cell lysates were prepared from SweAPP N2a cells treated with EGCG at 20 μM for different time points as indicated. (FIG. 3A) Western blot analysis by anti-TACE antibody shows TACE and cleaved fragments. (FIG. 3B) α-, β- and γ-secretase cleavage activities were analyzed in the cell lysates using secretase cleavage activity kits. Data are presented as percentage of fluorescence units/mg protein activated 1, 2 or 3 hours following EGCG treatment relative to control (PBS). A t-test revealed significant between α-secretase and either β-secretase or γ-secretase cleavage activity at 1, 2 and 3 hours after EGCG treatment (P<0.001). (FIGS. 3C, 3D, 3E) SweAPP N2a cells were treated with EGCG at 20 μM or PBS (control) in the presence or absence of TAPI-1 at various doses (FIG. 3C) or at 25 μM (FIGS. 3D, 3E) for 4 hours. Cell cultured supernatants were collected and cell lysates were prepared from these cultured cells. (FIG. 3C) Western blot analysis by antibody 369 shows holo APP, and two bands corresponding β-CTF and α-CTF. (FIG. 3D) Data are represented as percentage of Aβ secreted 4 hours after EGCG treatment in the presence or absence of TAPI-1 as indicated relative to control (PBS). A t-test revealed significant between EGCG and EGCG plus TAPI-1 treatment condition (P<0.001); reduction for each treatment condition is indicated. (FIG. 3E) α-secretase leavage activity is presented as percentage of fluorescence units/mg protein following EGCG treatment relative to control (PBS). A t-test revealed significant between EGCG treatment and co-treatment with EGCG and TAPI-1 (P<0.001); increased levels of activity are indicated.

FIGS. 4A-4F show EGCG in vivo treatment results in non-amyloidogenic APP processing. Brain homogenates were prepared from female 10-month-old transgenic APP_{sw} animals treated with EGCG (n=7) and PBS (n=5). (FIG. 4A, top) Western blot by antibody 369 shows holo APP, and two bands corresponding β-CTF and α-CTF. (FIG. 4A, middle, low) Western blot analysis by 22C11 shows holo APP (middle, following IP/anti-C-terminal APP) and sAPP-α (low, after IP/anti-C-terminal APP and IP again/6E10). Soluble Aβ (FIG. 4B) and insoluble Aβ prepared with 5 M guanidine (FIG. 4C) were analyzed by ELISA. Data are represented as mean±SEM of Aβ (pg/mg protein). For FIGS. 4B, 4C, A t-test revealed significant between EGCG- and PBS-treated transgenic APP_{sw} mice for either soluble or insoluble Aβ (p<0.001). (FIG. 4D) α-, β- and γ-secretase cleavage activities were analyzed by secretase cleavage activity kits. Data are presented as mean±SEM of fluorescence units/mg protein. A t-test revealed significant between EGCG- and PBS-treated transgenic APP_{sw} mice for α-secretase (P<0.001). (FIG. 4E) Mouse brain paraffin sections stained with anti-human Aβ antibody (4G8); left control PBS-treated mice. Right, EGCG-treated mice. Top, cingulated cortex (CC); Middle, hippocampus (H); Bottom, entorhinal cortex (EC). (FIG. 4D) Percentages of 4G8-positive Aβ plaques (mean±SEM) were calculated by quantitative image analysis; reduction for each brain region is indicated. A t-test for independent samples revealed significant differences between groups for each brain region examined in (FIG. 4D). (FIG. 4F) illustrates bar graphs comparing the Aβ burden (%) for transgenic APP_{sw} mice treated with two different compounds.

FIG. 5 illustrates various green tea polyphenols useful in the methods of the subject invention.

FIGS. 6A-6C show the treatment of SweAPP N2a cells with EGCG results in ADAM10 cleavage. FIG. 6A shows expression of ADAM9, 10, and 17 was analyzed in cell lysates from SweAPP N2a cells treated with EGCG at the various doses indicated for 8 h by Western blot (WB). Densitometry analysis shows the band density ratio of the mature (mADAM10) to the pro (pro-ADAM10) form of ADAM10 or the band density ratio of ADAM9 or 17 to actin as indicated in panels to the right. One-way ANOVA revealed significant differences between EGCG-treated cells and control cultures on the ratio of MADAM10 to pro-ADAM10 (**P<0.001; *P<0.05), but no significant differences were noted for the ratios of ADAM9 or 17 to actin (P>0.05). Similar data were obtained in three independent experiments. FIG. 6B shows ADAM10 mRNA level was analyzed in SweAPP N2a cells treated with EGCG at the various doses indicated for 8 h by RT-PCR. Densitometry analysis shows the band density ratio of ADAM10 to γ-actin as indicated in the panel below. One-way ANOVA revealed no significant differences between EGCG-treated cells and control cultures on the ratio of ADAM10 to γ-actin (P>0.05). Similar data were obtained in two independent experiments. FIG. 6C shows cell lysates were prepared from SweAPP N2a cells treated with EGCG (20 μM for 0, 30, 60, or 120 min and subjected to WB for ADAM10 cleavage analysis. Densitometry analysis shows the band density ratios of to pro-ADAM10 to actin and mADAM10 to actin as indicated in the panels to the right. One-way ANOVA revealed significant between-time points differences (**P<0.001; *P<0.05) with n=3 for each condition.

FIGS. 7A, 7B show EGCG treatment enhances ADAM10 in both cultured neuronal and microglial cells. In FIGS. 7A, 7B, cell lysates were prepared from N2a cells or N9 microglial cells (FIG. 7A) or wild-type mouse-derived primary neuronal or microglial cells (FIG. 7B) that were treated with EGCG at various doses as indicated for 8 h and subjected to WB for ADAM10 cleavage analysis. Densitometry analysis shows the band density ratio of mADAM10 to pro-ADAM10 as indicated below. One-way ANOVA followed by post-hoc analysis revealed significant differences between N2a and N9 cells treated with EGCG at 10 and 20 μM (**P<0.001), and primary neuronal and microglial cells treated with EGCG at 10 and 20 μM (**P<0.001). Similar results were obtained in two independent experiments.

FIGS. 8A-8H show EGCG-induced ADAM10 activation correlates with APP α-secretase cleavage in vitro. SweAPP N2a cells (FIGS. 8A, 8B) or Tg2576 mouse-derived primary neuronal cells (FIGS. 8E, 8F) were treated with EGCG at various doses for 8 h and subjected to WB for APP CTFs and ADAM10. As indicated in panels to the right, densitometry analysis shows the band density ratio of α-C-terminal fragment (α-CTF) to full length APP (holo APP) for (FIGS. 8A, 8E) or mADAM10 to pro-ADAM10 for (FIGS. 8B, 8F). One-way ANOVA revealed significant between-EGCG dose differences on both ratios of α-CTF to holo APP and mADAM10 to pro-ADAM10 (**P<0.001). Conditioned media were collected from SweAPP N2a cells (FIGS. 8c, 8d) or Tg2576 mouse-derived primary neuronal cells (FIGS. 8G, 8H) after EGCG treatment and subjected to WB for sAPP-α or Aβ ELISA. Data were represented as % change relative to control (medium from cultured SweAPP N2a cells or primary neuronal cells without any treatment). One-way ANOVA revealed significant between-EGCG dose differences in both ratio of sAPP-α to actin (**P<0.001) and reduction of Aβ₁₋₄₀ and Aβ₁₋₄₂ (**P<0.001). Similar results were observed in three independent experiments.

FIGS. 9A-9D show siRNA knock-down efficiency for ADAM10, 17 or 9 is confirmed by Western blot analysis. Expression of ADAM10 (FIG. 9A), 17 (FIG. 9B) or 9 (FIG. 9C) was analyzed by WB in cell lysates from SweAPP N2a cells transfected with siRNA targeting ADAM10, 17 or 9 at 24 or 48 h after transfection. Densitometry analysis shows the band density ratios of pro-ADAM10, ADAM17, ADAM9 to actin as indicated in the panels below. One-way ANOVA revealed significant differences between siRNA transfected cells and control cultures on the ratio of ADAM family to actin (**P>0.001). Similar data were obtained in three independent experiments. In FIG. 9D, expression of ADAM9 or 17 was analyzed by WB in cell lysates from SweAPP N2a cells transfected with siRNA targeting ADAM10 at 48 h after transfection. Densitometry analysis shows the band density ratios of pro-ADAM17 to actin or ADAM10 to actin as indicated in the panel below. One-way ANOVA revealed no significant differences between siRNA transfected cells and control cultures on the ratio of ADAM9 or ADAM17 to actin (P>0.05). Similar data were obtained in two independent experiments.

FIGS. 10A-10C show ADAM10 is required for EGCG-induced APP α-secretase cleavage. Cell lysates (FIG. 10A) and conditioned media (FIGS. 10B, 10C) were prepared and collected from SweAPP N2a cells transfected with ADAM9, 10, or 17 siRNA or non-targeting siRNA control (siRNA control) for 48 h and then treated with EGCG (20 μM) for 8 h. In FIG. 10A, cell lysates were subjected to WB for APP CTFs and ADAM10 cleavage analyses. Densitometry analysis shows the band density ratios of α-CTF to holo APP (upper right panel), pro-ADAM10 to actin (middle right panel) or mADAM10 to actin (lower right panel) as indicated. In FIG. 10B, cell culture media were subjected to WB for sAPP-α secretion. Densitometry analysis shows the band density ratios of sAPP-α to actin as indicated below. In FIG. 10C, cell culture media were subjected to Aβ ELISA. Data are represented as % change relative to control (medium from cultured SweAPP N2a cells without any treatment). A t test revealed a significant difference between ADAM10 siRNA and ADAM9 or ADAM17 siRNA or siRNA control (**P<0.001) on the ratios of α-CTF to holo APP, pro-ADAM10 to actin, or mADAM10 to actin, and reduction of sAPP-α and Aβ species as indicated. However, there were no significant differences between ADAM9 or ADAM17 siRNA and siRNA control by a t-test (P>0.05). Data are representative of three independent experiments.

### DETAILED DISCLOSURE OF THE INVENTION

The subject invention concerns materials and methods for treating or preventing a neurodegenerative condition or disease associated with β-amyloid peptide deposition in neural tissue in a person or animal by administering a therapeutically effective amount of a polyphenol, or an analog, isomer, metabolite, or prodrug thereof, that increases expression or activity of a protein that exhibits α-secretase activity. In one embodiment, the protein that exhibits α-secretase activity is ADAM10. Polyphenols contemplated within the scope of the methods of the invention include epigallocatechin-3-gallate (EGCG) and epicatechin (EC). In one embodiment, the neurodegenerative disease or condition to be treated is Alzheimer's disease. In one embodiment, the polyphenol increases the cleavage activity of the protein having α-secretase activity. In another embodiment, the polyphenol increases the expression of the gene encoding the protein and/or increases the amount of the protein produced or present in a cell.

The subject invention concerns methods for increasing the cleavage activity of α-secretase by administering to a person or animal an effective amount of at least one of the active compounds present in or derived from green tea, including (−)-epigallocatechin-3-gallate (EGCG) and epicatechin (EC) as well as their analogs, isomers, prodrugs, metabolites, or salts thereof. Advantageously, increasing α-secretase activity can be useful in preventing or treating a disease characterized by amyloid deposition in a patient. In one embodiment, the amyloid disease is Alzheimer's disease. In some instances, the patient may be asymptomatic of an amyloid disease. In some methods, the patient has environmental and/or genetic risk factors that indicate a susceptibility of developing an amyloid disease. In other methods, the patient has no risk factors.

In the methods of the subject invention, α-secretase levels and/or activity can be elevated enough to 1) reduce pathological levels of Aβ production to normal or nonpathological levels and/or 2) to increase sAPPα to levels that are neuroprotective in a mammalian patient.

Without being limited by theory, the increased efficiency of the α-secretase cleavage activity by following the methods of the subject invention may result from an up-regulation of tumor necrosis factor α-converting enzyme (TACE). Acts of TACE that are elicited by practicing the methods of the subject invention possibly promote α-secretase activity preferentially over TNF-α maturation and release. Another contribution, alone or in combination with the above, to the increased activity of the α-secretase may involve EGCG, EC, their analogs, metabolites, prodrugs, or salts thereof directly binding α-secretase active sites within the peptide Aβ12-22, thereby allowing TACE or additional α-secretase cleavages.

One aspect of the subject invention is directed to methods for treating elevated levels of amyloid peptides. Specifically, the subject methods can be used to reduce β-amyloid (Aβ) generation within a cell in vivo or in vitro. Advantageously, the methods of the subject invention also enhance the cleavage of tumor necrosis factor α-converting enzyme (TACE).

Advantageously, EGCG and EC from green tea or other plants or from synthetic sources greatly promotes formation of alpha C-terminal fragment (CTF) of amyloid precursor protein (APP) and secreted APP-alpha (sAPPα) via increased activity of α-secretase. Purified extracts of natural compounds (EGCG and EC) from green tea have a significant effect on APP metabolism in a dose-dependent manner in APP_{sw}-transfected N2a cells, as evidenced by markedly decreased levels of Aβ release (including 1-40 and 1-42) in cultured media by Aβ ELISA. These effects are significantly correlated with increased secreted APP-alpha and cell lysated-derived alpha-CTF. Furthermore, there are not any changes in holo APP expression as examined by Western blot. Most importantly, this effect is dependent on increased alpha-secretase activity.

EGCG, a flavonoid found in green tea, significantly reduces Aβ generation in N2a neuroblastoma cells overexpressing human amyloid precursor protein (APP). This effect is supported by: 1) a markedly increased cleavage of α-C-terminal fragment of APP (α-CTF) and secretion of APP-α; 2) enhanced cleavage of tumor necrosis factor α-converting enzyme (TACE) and elevated α-secretase cleavage activity. Furthermore, EGCG treated transgenic mice overproducing Aβ show decreased Aβ₁₋₄₂ level and Aβ plaque load associated with increased generation of non-amyloidogenic APP fragments (α-CTF and sAPP-α) and elevated activity of α-secretase cleavage in the brain.

The extracts, compounds or combination of compounds derived from green tea that are useful in the subject invention are generally prepared by methods known in the art. Tea extracts containing high concentrations of EGCG and other naturally occurring tea-derived polyphenols are commercially available. With regard to chemical synthesis of the compounds, reference is made to Li et al., (2001), which is incorporated in its entirety by reference.

While the invention is described with respect to tea-derived polyphenol compounds or analogs, from this disclosure the skilled artisan will appreciate and envision synthetic routes to obtain and/or prepare the active compounds, including synthetic tea polyphenols and their derivates. The compounds utilized in the subject methods can be derived from green tea or other plant or food products or can be produced synthetically. Analogs of green tea extracts useful in the compositions and methods of the subject application are known in the art and examples are described in U.S. Pat. No. 6,713,506; Lam, W. H. et al., (2004); Smith, D. M. et al., (2004); and Wan, S. B. et al., (2005), all of which are incorporated in their entirety by reference. As discussed below, analogs useful in increasing α-secretase cleavage activity should not be analogs to gallocatechin and/or (−)catechin, two green the flavonoids that decrease α-secretase activity.

Each green tea derived polyphenol administered in the methods of the subject invention may also be administered as a drinkable tea. The tea may be purified by removal of compounds known to antagonize α-secretase activity including, for example, (−)-gallocatechin (GC) and (−)-catechin (C).

The methods of the subject invention may also be practiced by administering pharmaceutical compositions to a patient. The pharmaceutical compositions comprise at least one active ingredient in one or more pharmaceutically acceptable carriers. Each carrier must be acceptable in the sense of being compatible with the other ingredients of the formulation and hot injurious to the patient. One such composition comprises EGCG, EC, or pharmaceutically acceptable salts, or analogs thereof, or a mixture of any of the foregoing in a pharmaceutically acceptable carrier.

Formulations include those suitable for oral, rectal, nasal, topical (including transdermal, buccal and sublingual), vaginal, parental (including subcutaneous, intramuscular, intravenous and intradermal) and pulmonary administration. The formulations can conveniently be presented in unit dosage form and can be prepared by any methods well known in the art of pharmacy. Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then if necessary shaping the product. Formulations of the subject invention suitable for oral administration can be presented as discrete units such as capsules, cachets or tablets, each containing a predetermined amount of the active ingredient; or as an oil-in-water liquid emulsion, water-in-oil liquid emulsion or as a supplement within an aqueous solution, for example, a tea. The active ingredient can also be presented as bolus, electuary, or paste.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; mouthwashes comprising the active ingredient in a suitable liquid carrier; and chocolate comprising the active ingredients.

Pharmaceutical compositions for topical administration according to the subject invention can be formulated as an ointment, cream, suspension, lotion, powder, solution, paste, gel; spray, aerosol or oil. Alternatively, a formulation can comprise a patch or a dressing such as a bandage or adhesive plaster impregnated with active ingredients, and optionally one or more excipients or diluents. Topical formulations preferably comprise compounds that facilitate absorption of the active ingredients through the skin and into the bloodstream.

Formulations suitable for nasal administration, wherein the carrier is a solid, include a coarse powder having a particle size, for example, in the range of about 20 to about 500 microns, which is administered in the manner in which snuff is taken, i.e., by rapid inhalation through the nasal passage from a container of the powder held close up to the nose. Suitable formulations wherein the carrier is a liquid for administration by nebulizer, include aqueous or oily solutions of the agent.

Formulations suitable for parenteral administration include aqueous and non-aqueous isotonic sterile injection solutions which can contain antioxidants, buffers, bacteriostats and solutes which render the formulation isotonic with the blood of the intended recipient; and aqueous and non-aqueous sterile suspensions which can include suspending agents and thickening agents, and liposomes or other microparticulate systems which are designed to target the compound to blood components or one or more organs. The formulations can be presented in unit-dose or multi-dose or multi-dose sealed containers, such as for example, ampoules and vials, and can be stored in a freeze-dried (lyophilized) condition requiring only the addition of the sterile liquid carrier, for example, water for injections, immediately prior to use. Extemporaneous injection solutions and suspensions can be prepared from sterile powders, granules and tablets of the kind previously described.

Preferred unit dosage formulations are those containing a daily dose or unit, daily subdose, as herein above-recited, or an appropriate fraction thereof, of an agent. It should be understood that in addition to the ingredients particularly mentioned above, the formulations useful in the present invention can include other agents conventional in the art regarding the type of formulation in question. For example, formulations suitable for oral administration can include such further agents as sweeteners, thickeners, and flavoring agents. It also is intended that the agents, compositions, and methods of this invention be combined with other suitable compositions and therapies.

Various delivery systems are known in the art and can be used to administer a therapeutic agent of the invention, e.g., encapsulation in liposomes, microparticles, microcapsules, receptor-mediated endocytosis and the like. Methods of delivery include, but are not limited to, intra-arterial, intramuscular, intravenous, intranasal, and oral routes. In a specific embodiment, the pharmaceutical compositions of the invention can be administered locally to the area in need of treatment; such local administration can be achieved, for example, by local infusion during surgery, by injection, or by means of a catheter.

Therapeutic amounts can be empirically determined and will vary with the pathology being treated, the subject being treated, and the efficacy and toxicity of the agent. Similarly, suitable dosage formulations and methods of administering the agents can be readily determined by those of skill in the art.

The pharmaceutical compositions can be administered by any of a variety of routes, such as orally, intranasally, parenterally or by inhalation therapy, and can take form of tablets, lozenges, granules, capsules, pills, ampoule, suppositories or aerosol form. They can also take the form of suspensions, solutions, and emulsions of the active ingredient in aqueous or nonaqueous diluents, syrups, granulates or powders. In addition to a compound of the subject invention, the pharmaceutical compositions can also contain other pharmaceutically active compounds or a plurality of compounds of the invention.

Ideally, the compound should be administered to achieve peak concentrations of the active compound at sites of the disease. Peak concentrations at disease sites can be achieved, for example, by intravenously injecting of the agent, optionally in saline, or orally administering, for example, a tablet, capsule or syrup containing the active ingredient.

The compositions can be administered simultaneously or sequentially with other drugs or biologically active agents. Examples include, but are not limited to, antioxidants, free radical scavenging agents, peptides, growth factors, antibiotics, bacteriostatic agents, immunosuppressives, anticoagulants, buffering agents, anti-inflammatory agents, anti-pyretics, time-release binders, anesthetics, steroids and corticosteroids. In one specific embodiment, the compositions are administered simultaneously or sequentially with galantamine, deprenyl, cdp choline, folate, Vitamin B12, Vitamin B6, piracetam, vinpocetine, idebenone, pyritinol, memantine, or a combination of any of the forgoing.

Another aspect of the subject application is directed to promoting Alzheimer's progression by inhibiting α-secretase activity. These methods are useful, for example, in approximating Alzheimer's disease in cell lines or animal models. Other purified green tea extracts, including gallocatechin, gallic acid, catechin, their analogs, prodrugs, metabolites, and salts are useful in attenuating the beneficial effects of ECGC and EC.

Without being limited by theory, if up regulating TACE is the mechanism by which α-secretase activity is affected by green tea flavonoids, these methods could also be used to treat or prevent inflammatory or auto-immune diseases of the peripheral nervous system e.g., rheumatoid arthritis, autonomic neuropathy, brachial plexus injuries, cervical radiculopathy, chronic inflammatory demyelinary polyneuropathy, diabetic neuropathies, dysautonomia, erb-duchenne palsy, dejerine-klumke palsy, glossopharyngeal neuralgia, hereditary neuropathies, Isaac's syndrome, and postherpetic neuralgia, or any disease characterized by up-regulated TACE activity. These methods comprise administering a physiological effect in amounts of GA, GC, C, their analogs, salts, metabolites, prodrugs or a combination of the foregoing. In another embodiment, these compounds can be administered as a pharmaceutical composition comprising GA, GC, C, their analogs, salts, metabolites, prodrugs or a combination of the foregoing in a pharmaceutical composition.

For the purpose of this invention the following terms are defined below:

It will be understood that a specific “effective amount” for any particular in vivo or in vitro application will depend upon a variety of factors including the activity of the specific compound employed, the age, body weight, general health, sex, and/or diet of the individual, time of administration, route of administration, rate of excretion, drug combination and the severity of the particular disease undergoing prevention or therapy. For example, the “effective amount” may be the amount of compound of the invention necessary to achieve increased α-secretase activity in vivo or in vitro. The “effective amount” may be the amount of compound of the invention necessary to enhance the cleavage of tumor necrosis factor α-converting enzyme.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include citric acid, lactic acid, tartaric acid, fatty acids, and the like. Salts may also be formed with bases. Such salts include salts derived from inorganic or organic bases, for example alkali metal salts such as magnesium or calcium salts, and organic amine salts such as morpholine, piperidine, dimethylamine or diethylamine salts.

As used herein, the term “pharmaceutically acceptable carrier” includes any and all solvents (such as phosphate buffered saline buffers, water, saline), dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in therapeutic compositions is contemplated. Supplementary active ingredients can also be incorporated into the compositions. The pharmaceutical compositions of the subject invention can be formulated according to known methods for preparing pharmaceutically useful compositions. Formulations are described in a number of sources which are well known and readily available to those skilled in the art. For example, Remington's Pharmaceutical Science (Martin, E. W., 1995) describes formulations which can be used in connection with the subject invention.

The subject invention also concerns methods for screening for candidate drugs or compounds that can be used to treat or prevent a neurodegenerative disease or condition in a person or animal. In one embodiment, a candidate drug or compound is assayed to determine if it can increase expression or activity of an α-secretase enzyme. In a specific embodiment, the α-secretase enzyme is ADAM10. In a specific embodiment, cells that produce β-amyloid peptides are contacted with a candidate drug or compound and then assayed to determine whether the levels of β-amyloid peptides are decreased. In an exemplified embodiment, the cells are neuroblastoma cells that overproduce β-amyloid peptide and the β-amyloid peptide is Aβ₁₋₄₂ or Aβ₁₋₄₀. In one embodiment, the β-amyloid peptide is overexpressed by the cell. In a further embodiment, the cells overproduce or express elevated levels of an APP protein. In one embodiment, the APP protein is a mutant protein. In another embodiment, the cells are neuronal cells from an animal. In a specific embodiment, the animal has a pathological condition that is the same as or similar to Alzheimer's disease. In an exemplified embodiment, the neuronal cells are from transgenic mice that overexpress an APP protein. In a specific embodiment, the APP protein is a mutant APP protein and the transgenic mice are APP_{sw} line 2576.

Reference herein to increased expression or activity of an α-secretase enzyme refers to any form of increase in expression or activity, including, but not limited to an increase in transcription of a gene encoding an enzyme with α-secretase activity; an increase in half-life of an RNA molecule encoding the enzyme; an increase in translation of the RNA into a protein having α-secretase activity, an increase in the half-life of the protein having α-secretase activity, and any other means that results in an increase in the amount of protein produced or present in the cell; or an increase in the enzymatic activity of the protein having α-secretase activity.

The subject invention also concerns compositions comprising a polyphenol of the invention in a pharmaceutically acceptable carrier or diluent.

The subject invention also concerns compositions comprising polyphenols that increase expression or levels of a protein having α-secretase activity, such as ADAM10, and agents or compounds that inhibit or decrease expression or levels of protein having β-secretase activity or γ-secretase activity. In one embodiment, the polyphenols are EGCG and/or EC, and analogs, isomers, metbolites, or prodrugs thereof. Preferably, the polyphenols are provided in purified form. More preferably, the polyphenols are purified to a level wherein compounds that antagonize the activity of the polyphenols are removed or decreases to a level wherein they do not antagonize the action of the polyphenols. In one embodiment, the agents or compounds comprise nucleic acid that is antisense to nucleic acid encoding a protein with β-secretase activity or γ-secretase activity, and/or comprise a small interfering RNA molecule that interferes with expression of a protein having β-secretase activity or γ-secretase activity.

As used herein, the terms “individual” and “patient” are used interchangeably to refer to any vertebrate, mammalian species, such as humans and animals. Mammalian species which benefit from the disclosed methods of treatment include, and are not limited to, apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales. Human or non-human animal patients can range in age from neonates to elderly.

The term “administering” and “administration” is intended to mean a mode of delivery including, without limitation, oral, rectal, parenteral, subcutaneous, intravenous, intramuscular, intraperitoneal, intraarterial, transdermally or via a mucus membrane. The preferred one being orally. One skilled in the art recognizes that suitable forms of oral formulation include, but are not limited to, a tablet, a pill, a capsule, a lozenge, a powder, a sustained release tablet, a liquid, a liquid suspension, a gel, a syrup, a slurry, a suspension, and the like. For example, a daily dosage can be divided into one, two or more doses in a suitable form to be administered at one, two or more times throughout a time period.

The term “therapeutically effective” is intended to mean an amount of a compound sufficient to substantially improve some symptom associated with a disease or a medical condition. For example, in the treatment of cancer, a compound which decreases, prevents, delays, suppresses, or arrests any symptom of the disease would be therapeutically effective. A therapeutically effective amount of a compound is not required to cure a disease but will provide a treatment for a disease such that the onset of the disease is delayed, hindered, or prevented, or the disease symptoms are ameliorated, or the term of the disease is changed or, for example, is less severe or recovery is accelerated in an individual.

The term “analog” is intended to mean a compound that is similar or comparable, but not identical, to a reference compound, i.e. a compound similar in function and appearance, but not in structure or origin to the reference compound. For example, the reference compound can be a reference green tea polyphenol and an analog is a substance possessing a chemical structure or chemical properties similar to those of the reference green tea polyphenol. As used herein, an analog is a chemical compound that may be structurally similar to another but differs in composition (as in the replacement of one atom by an atom of a different element or in the presence of a particular functional group). An analog may be extracted from a natural source or be prepared using synthetic methods.

The terms “treatment”, “treating” and the like are intended to mean obtaining a desired pharmacologic and/or physiologic effect, e.g., increasing activity of α-secretase. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, particularly a human, and includes: (a) preventing a disease or condition (e.g., preventing amyloid disease) from occurring in an individual who may be predisposed to the disease but has not yet been diagnosed as having it; (b) inhibiting the disease, (e.g., arresting its development); or (c) relieving the disease (e.g., reducing symptoms associated with the disease).

As used in this specification, the singular forms “a”, “an”, and “the” include plural references unless the context clearly dictates otherwise.

The terms “comprising”, “consisting of”, and “consisting essentially of” are defined according to their standard meaning and may be substituted for one another throughout the subject application in order to attach the specific meaning associated with each term.

### Materials and Methods for Examples 1-4

### Reagents

Green tea derived flavonoids (>95% HPLC), including EGCG, (−) EC, (+) EC, GC and C were purchased from Sigma Chemical Co. (St Louis, Mo.). TAPI-1, β and γ-secretase inhibitors were obtained from Calbiochem (San Diego, Calif.). Green tea extract (75% polyphenols) was obtained from the Vitamin Shoppe™ (North Bergen, N.J.).

### ELISA

Cultured cells were lysed in ice-cold-lysis buffer (20 mM Tris, pH 7.5, 150 mM NaCl, 1 mM EDTA, 1 mM EGTA, 1% v/v Triton X-100, 2.5 mM sodium pyropgosphate, 1 mM β-glycerolphosphate, 1 mM Na₃VO₄, 1 μg/mL leupeptin, 1 mM PMSF) as previously described (Tan et al., 2002). Mouse brains were isolated under sterile conditions on ice and placed in ice-cold lysis buffer. Brains were then sonicated on ice for approximately 3 min, allowed to stand for 15 min at 4° C., and centrifuged at 15,000 rpm for 15 min. Total Aβ₁₋₄₂ species were detected by acid extraction of brain homogenates in 5 M guanidine buffer (ref), followed by a 1:10 dilution in lysis buffer. Soluble Aβ₁₋₄₂ was directly detected in cultured cell lysates or brain homogenates prepared with lysis buffer described above by a 1:4 or 1:10 dilution, respectively. Aβ₁₋₄₂ was quantified in these samples using the Aβ₁₋₄₂ ELISA kit (BioSource, Camarillo, Calif.) in accordance with the manufacturer's instructions, except that standards included a 0.5 M guanidine buffer.

### Western Blot/Immunoprecipitation

Cultured cells and mouse brain were lysed in ice-cold lysis buffer described above, and an aliquot corresponding to 50 μg of total protein was electrophoretically separated using 16.5% Tris-tricine gels. Electrophoresed proteins were then transferred to PVDF membranes (Bio-Rad), washed in dH₂O, and blocked for 1 hour at ambient temperature in Tris-buffered saline (TBS; Bio-Rad) containing 5% (w/v) for non-fat dry milk. After blocking, membranes were hybridized for 1 hour at ambient temperature with various antibodies, including against the C- (369), N-terminus of APP (22C11), the N-terminus of Aβ (6E10), and ADAM10 and TACE (Calbiochem). Membranes were then washed 3× or 5 min each in dH₂O and incubated for 1 hour at ambient temperature with the appropriate HRP-conjugated secondary antibody (1:1,000, Pierce Biotechnology, Inc. Rockford, Ill.). All antibodies were diluted in TBS containing 5% (w/v) of non-fat dry milk. Blots were developed using the luminal reagent (Pierce Biotechnology). Densitometric analysis was done using the Fluor-S Multimager™ with Quantity One™ software (Bio-Rad). Immunoprecipitation was performed for detection of sAPP-α, sAPP-β and Aβ by incubating 200 μg of total protein of each sample with 6E10 (1:100; Signet) or 22C11 (1:100; Roche, Basel, Switzerland) overnight with gentle rocking at 4° C., and 10 μL of 50% protein A-Sepharose beads were then added to the sample (1:10; Sigma) prior to gentle rocking for an additional 4 hours at 4° C. Following washes with 1× cell lysis buffer, samples were subjected to Western blot as described above. Antibodies used for Western blot included antibody 369 (1:1,000), anti-C-terminal APP antibody (1:500; Chemicon, Temecula, Calif.), BAM-10 (1:1,000; Sigma), 6E10 (1:1,000; Signet) or anti-Actin antibody (1:1,500; as an internal reference control; Roche). α-, β- and γ-secretase activities were quantified in cell lysates and mouse brain homogenates using available kits based on secretase-specific peptides conjugated to fluorogenic receptor molecules (R&D Systems, Minneapolis, Minn.).

### Mice

Transgenic APP_{sw} mice (the line 2576) (Hsiao K, et al., 1996) were purchased from Taconic (Germantown, N.Y.). For IP route, a total of 12 female transgenic-APP_{sw} mice were used in this study; 7 mice received EGCG, and the other 5 received PBS. Beginning at 12 months of age, transgenic APP_{sw} mice were IP injected with EGCG (20 mg/kg) or PBS daily for 60 days based on previously described methods (Chyu et al., 2004). These mice were then sacrificed at 14 months of age for analyses of Aβ levels and Aβ load in the brain (Tan et al., 2002). For ICV route (n=3; female), mice were ICV injected with EGCG [(5 μL (10 μg)/mouse)] or PBS once (Tan et al., 2002 and Siegel, 2003). 24 hours after injection, these mice were sacrificed for analysis of cerebral Aβ levels (Tan et al., 2002). Animal were housed and maintained in the College of Medicine Animal Facility at the University of South Florida (USF), and all experiments were in compliance with protocols approved by the USF Institutional Animal Care and Use Committee.

### Immunochemistry Analysis

Mice were anethetized with isofluorane and transcardinally perfused with ice-cold physiological saline containing heparin. Brains were isolated and halved (such that one half was used for immunochemistry analysis and the remaining half was used for preparation of homogenates). Brains were then fixed in 4% paraformaldehyde at 4° C. overnight and routinely processed in paraffin by a core facility at the Department of Pathology (USF College of Medicine). Using a microtome, five 5-μm sections were cut from brains (150 μm apart). Immunohistochemical staining was performed in accordance with the manufacture's instructions using the VECTATAIN Elite™ ABC kit (Vector Laboratories, Burlingame, Calif.), using anti-human amyloid-β antibody (clone 4G8, 1:100; Signet, Dedham Mass.). Images were obtained using an Olympus BX51 microscope and digitized using an attached MagnaFire™ imaging system (Olypus, Tokyo, Japan). Aβ burden was determined in transgenic APP_{sw} mouse brains by quantitative image analysis. Briefly, images of five 5-μm sections (150 μm apart) through each anatomic region of interest (hippocampus or cortical areas) were captured and a threshold optical density was obtained that discriminated staining form background. Manual editing of each field was used to eliminate artifacts. Data are reported as percentage of immunolabeled area captured (positive pixels) divided by the fill area captured (total pixels). Quantitative image analysis was performed by a single examiner (TM) blinded to sample identities.

### Example 1

### Green Tea Polyphenol EGCG Markedly Inhibits Aβ₁₋₄₂ Production in Murine N2A Cells Transfected with Human Wild-Type APP695 and Transgenic APP_{sw} Mouse-Derived Primary Neuronal Cells

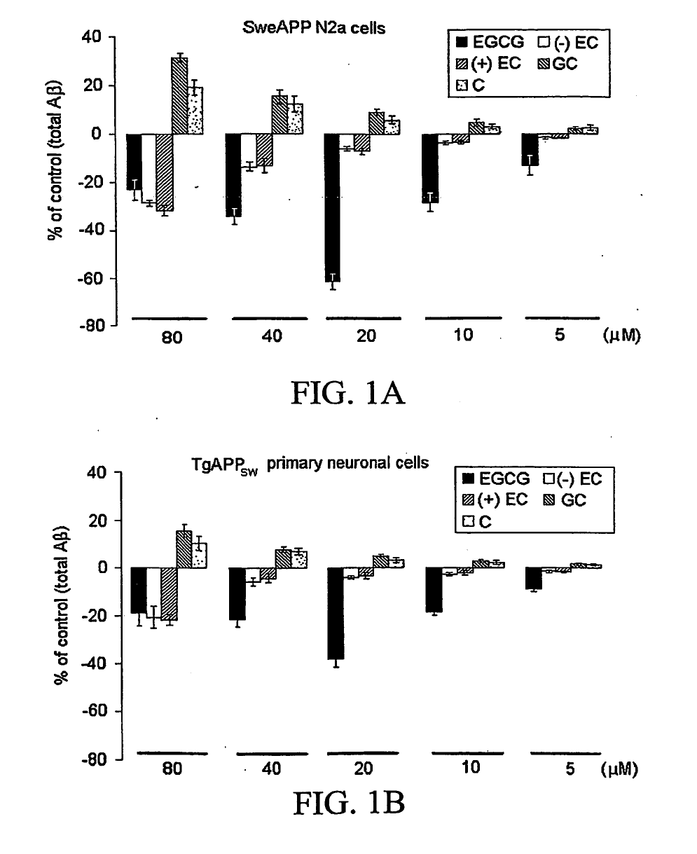

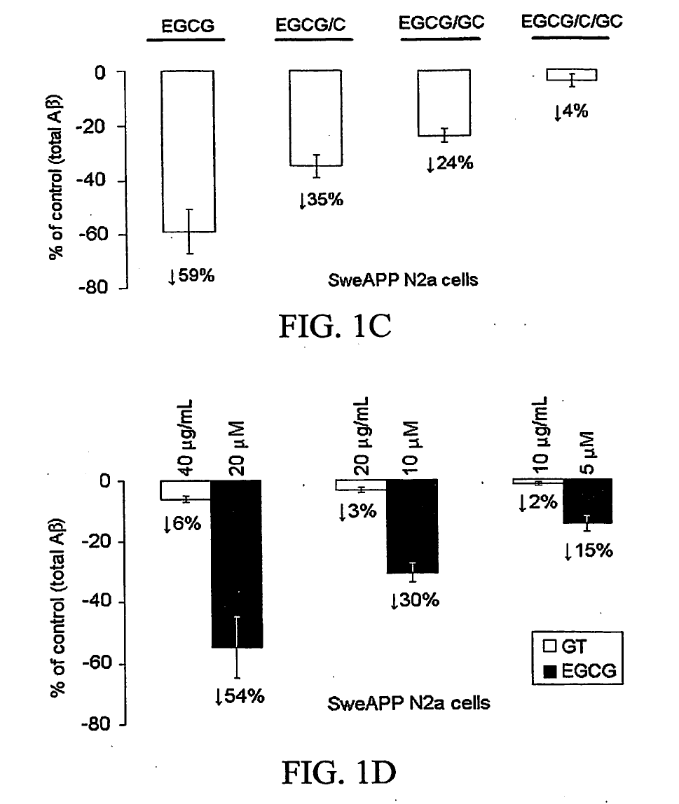

The HPLC analysis of green tea shows that EGCG is the major component along with others, including (−)-epicatechin [(−) EC], (+)-epicatechin [(+) EC], (−)-gallocatechin (GC) and (−)-catechin (C). In order to examine the effects of green tea's components on APP cleavage processing, for a first step, N2a neuroblastoma cells overproducing Aβ were treated with each of the aforementioned components at a wide range of doses. In addition, doses of (+)-catechin hydrate (CH) and gallic acid monohydrate (GAM) were included in the first step. EGCG markedly reduced Aβ₁₋₄₂ generation in either human wild-type APP695-transfected N2a cells or primary transgenic APP_{sw}-derived neuronal cells in a dose-dependent manner (FIGS. 1A and 1B). Most importantly, at 20 μM, EGCG treatment reduced Aβ₁₋₄₂ generation in both human APP695-transfected N2a cells by 60% (FIG. 1A) and primary transgenic APP_{sw}-derived neuronal cells by 40% (FIG. 1b) over control (PBS). Interestingly, either (−) EC or (+) EC only inhibited Aβ₁₋₄₂ production in both of cultured neuronal cells by nearly 20-30% at a relative high dose (FIGS. 1A and 1B).

However, both GC and C significantly promote Aβ production by approximately 20-30% (for SweAPP N2a cells), 10-15% (for primary cultured cells) at 80 μM. Furthermore, to test whether GC and/or C could oppose the inhibition of Aβ generation by EGCG, SweAPP N2a cells were co-treated with EGCG (20 μM) and GC (80 μM), C (80 μM) or both for 12 hours. As expected, data show that the presence of GC or C, specifically the combination of both markedly inhibits the ability of EGCG to reduce Aβ production in SweAPP N2a cells (FIG. 1C). Without being limited by theory, it is hypothesized that EGCG individually is more capable of reducing Aβ generation in vitro than its mixture form. To further address this hypothesis, SweAPP N2a cells were incubated with both EGCG in its naturally occurring mixture form (as green tea extract, GT) at various doses and with equivalent doses of purified EGCG alone. As shown in FIG. 1D, data indicate that a more profound effect on the reduction of Aβ generation is elicited by the various levels of EGCG alone. In other words, the ability of the purified EGCG to inhibit Aβ generation is much greater than that of the same amounts EGCG present in GT mixtures.

### Example 2

### Analysis of Alteration of CTFS and Secreted Forms of APP in Human Wild-Type APP695-Transfected N2A Cells After EGCG Treatment

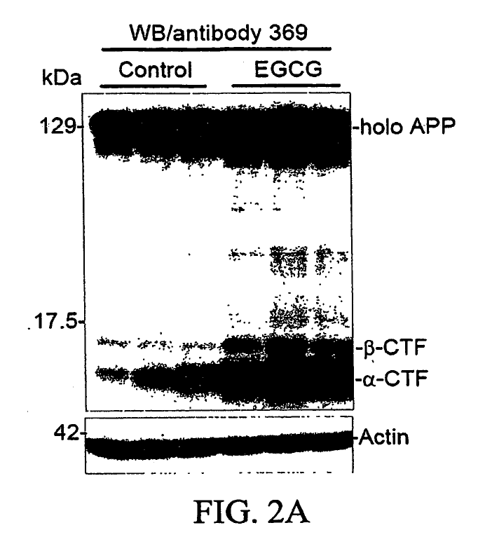

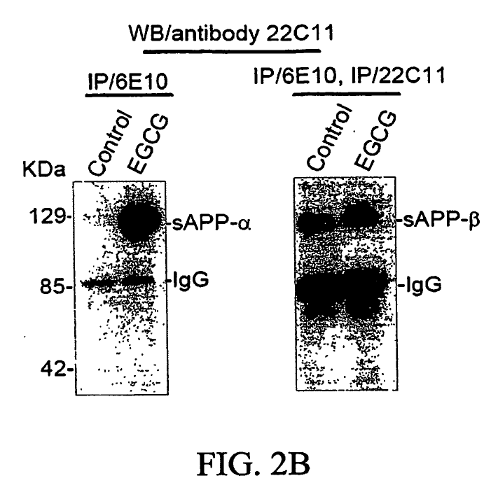

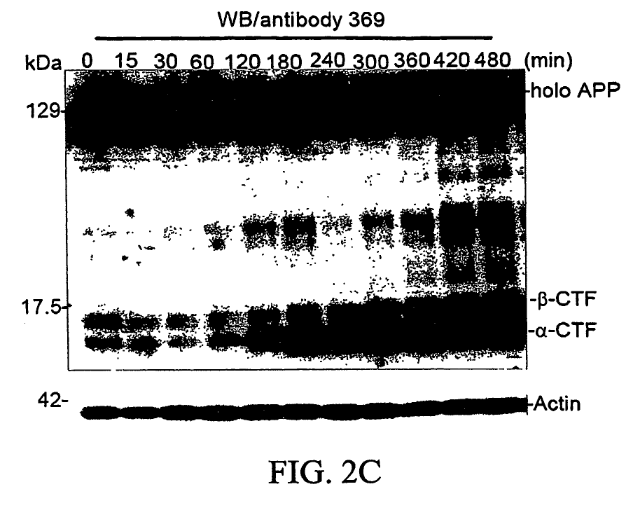

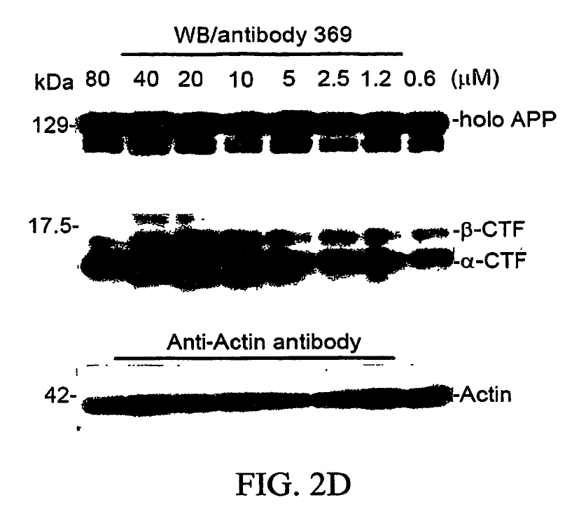

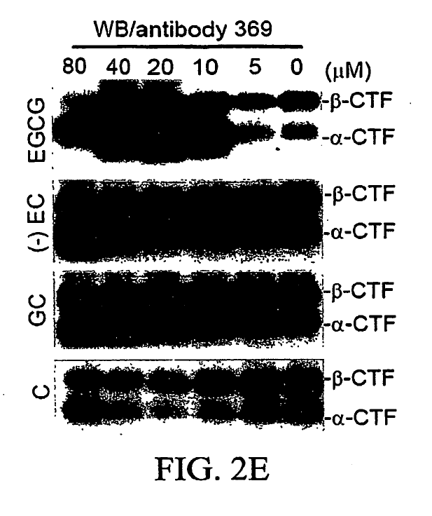

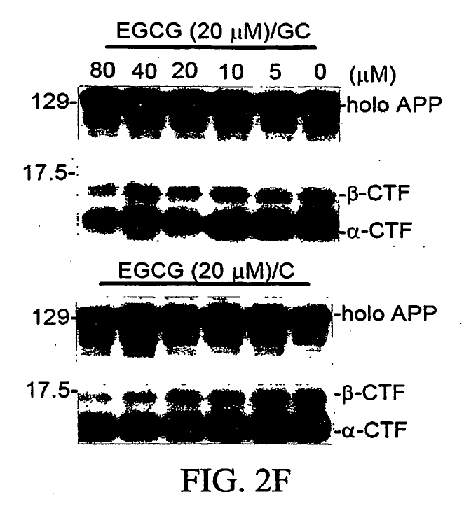

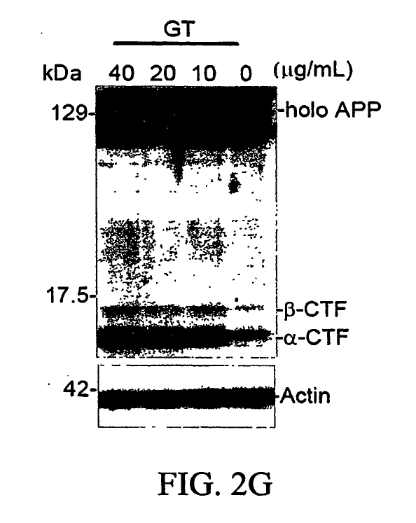

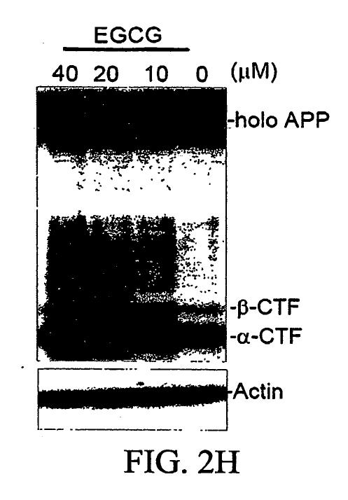

To unravel the underlying mechanisms, APP cleavages following EGCG treatment in human APP695-transfected N2a cells were examined using Western immunoblotting and immunoprecipitation. As expected, EGCG treatment results in a greatly increased α-CTF generation and ratio of α-CTF to β-CTF band density in a dose-dependent manner (FIG. 2A). In addition, treatment with either (−) EC only or (+) EC only increases α-CTF production at the high dose (FIGS. 2A and 2B). Furthermore, there are not any changes in total APP and holo APP expression in cells treated with EGCG treated condition (FIGS. 2B and 2C), suggesting that this treatment promotes APP α-cleavage rather than stimulates the expression of APP. Accordingly, the secreted form of APP-α (sAPP-α) was markedly increased in the cell cultured medium following EGCG treatment at 20 μM for 18 hours (FIG. 2D), which is strongly correlated with increased α-CTF generation in the cell lysates (FIG. 2C). Together, the results of these experiments further show that the treatment of EGCG favors non-amyloidogenic processing of APP in vitro.

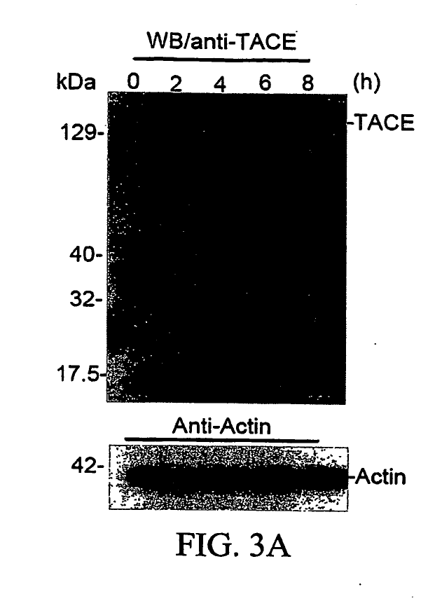

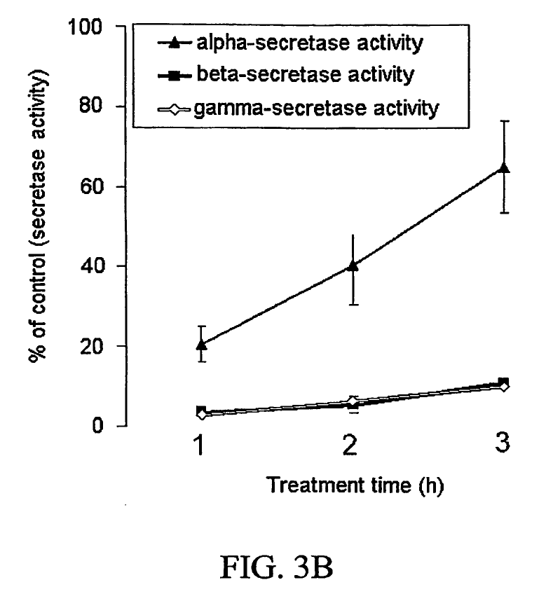

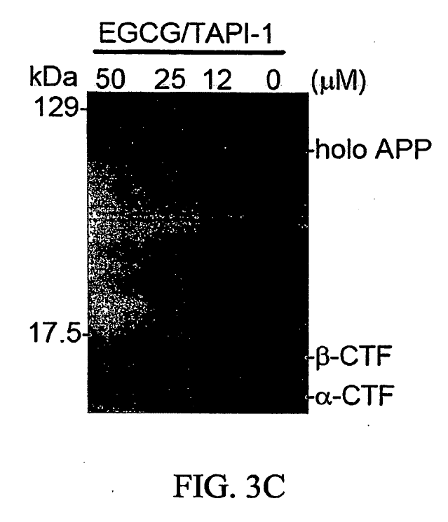

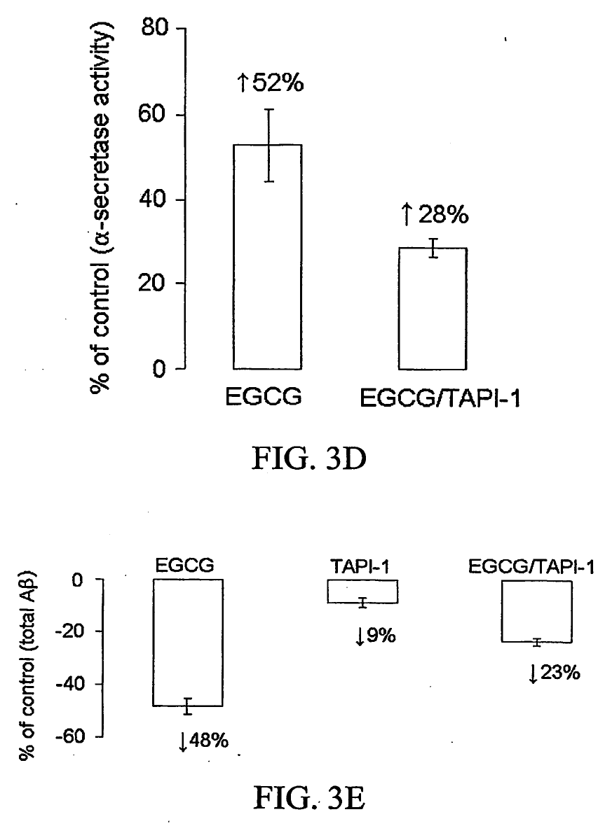

Most notably, these effects are in a time- and dose-dependent manner (FIGS. 2C and 2D). Additionally, as shown FIG. 2E (second panel) both (−) EC and (+) EC treatment only increase α-CTF generation at high doses [(+) EC data not shown)]. In contrast, both GC and C treatment result in decreased α-CTF generation and α-CTF to β-CTF ratio at, 80 μM as indicated (FIGS. 2E, third and fourth panels). More importantly, at these doses, either GC or C significantly opposes the effect of EGCG on α-CTF cleavage (FIG. 2F). In support of Aβ ELISA data as shown in FIG. 1D, EGCG in a purified form has a great effect of producing α-CTF generation than GT as given an equal amount of EGCG (FIGS. 2G and 2H). In addition to these effects, we did not observe any changes in total APP (data not shown) or holo APP expression during these treatment conditions, which was confirmed by Western blot analysis as shown in FIG. 2 (except FIG. 2B) and FIG. 3A. Thus, these treatments modulate APP cleavage rather than stimulate APP expression. Taken together, results of these experiments further demonstrate that EGCG favors non-amyloidogenic processing of APP in SweAPP N2a cells.

### Example 3

### Characterization of EGCG-Promoted α-Secretase Cleavage of APP in Human Wild-Type APP695-Transfected N2S Cells

As shown in FIG. 2C, Western immunoblotting analysis clearly shows a time dependent pattern of APP α-CTF generated by EGCG-treated human APP695-transfected N2a cells. Most notably, α-CTF generation is drastically increased in 3 to 4 hours and then through to 8 hours after EGCG treatment (FIG. 2C). To test whether the alteration of APP α-CTF could correlate with α-secretase cleavage activity, for the first step, the expression of TNF-α converting enzyme (TACE), one of candidates for cα-secretase cleavage of APP, following EGCG treatment at the same point was examined using Western immunoblotting. Results show that TACE expression is significantly increased in 3 to 4 hours following EGCG treatment and then rapidly cleaved through 8 hours (FIG. 3A). Furthermore, α-secretase cleavage activity in the cell lysate prepared from EGCG-PBS-treated human APP695-transfected N2a cells was directly measured. Results reveal that α-secretase cleavage activity is markedly elevated at the first 1 to 3 hours in human APP695-transfected N2a cells treated with EGCG (FIG. 3B). Taken together, these data demonstrate that elevated TACE cleavage activity plays a major role in promotion of α-secretase cleavage of APP in EGCG-treated human APP695-transfected N2a cells.

### Example 4

### In Transgenic APP_{sw} Mice, EGCG In Vivo Treatment Results in Non-Amyloidogenic APP Processing

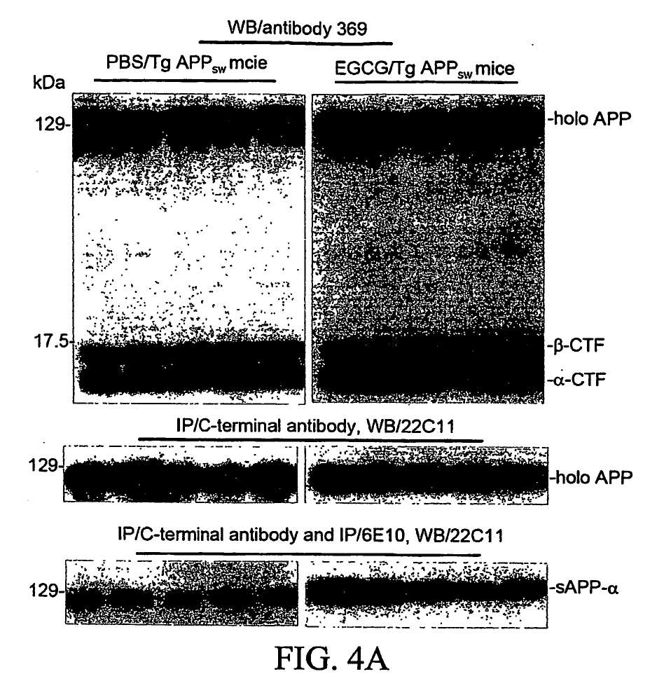

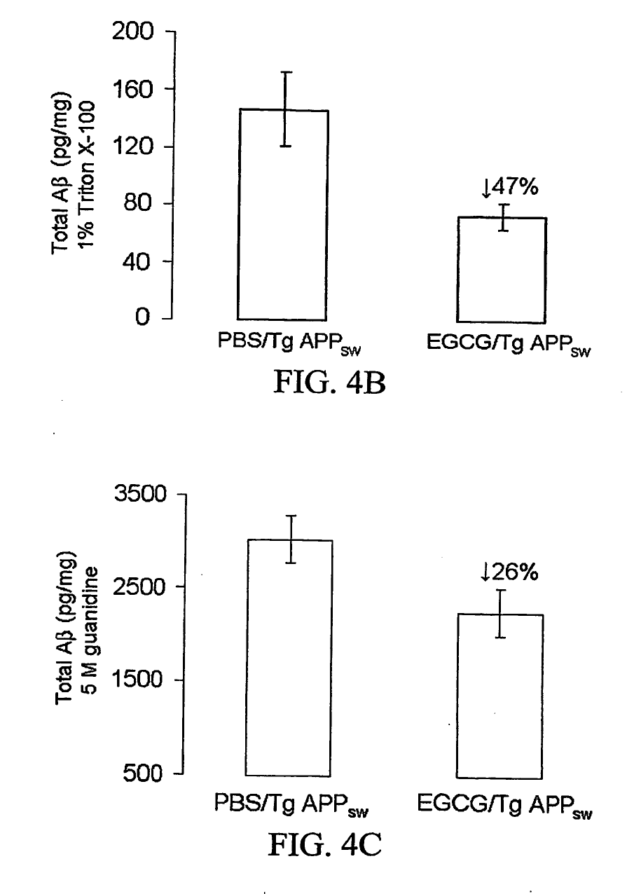

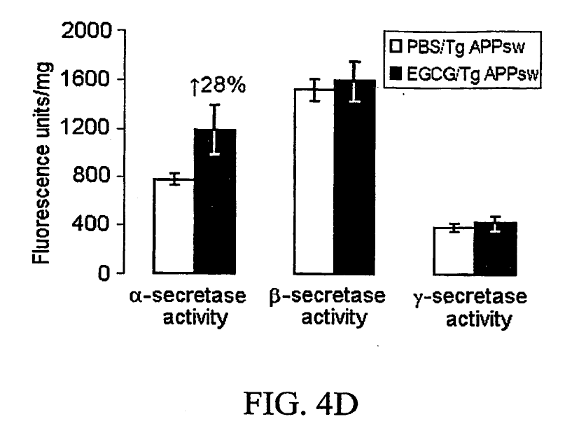

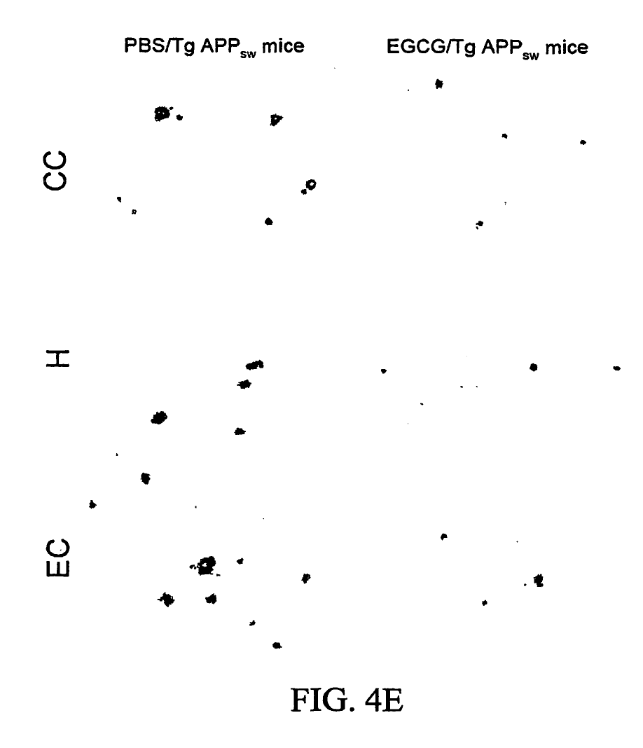

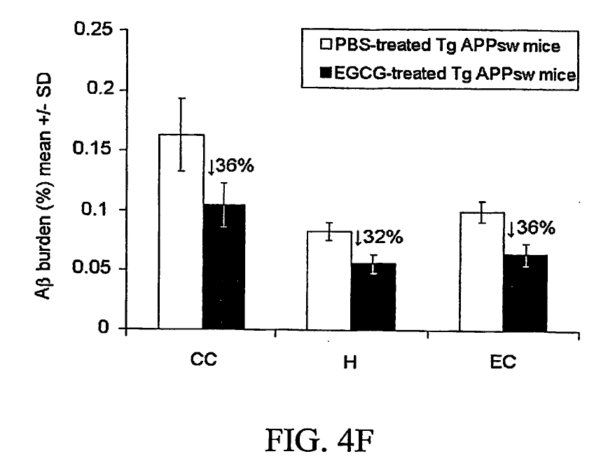

In accordance with these in vitro findings above, EGCG in vivo treatment was tested to determine if it could promote non-amyloidogenic APP processing and impact the Aβ levels/β-amyloid load in the brain of transgenic APP_{sw} mice. EGCG was administered to transgenic APP_{sw} mice, a transgenic mouse model of AD. EGCG was administered based on a treatment schedule that produces the benefit in ischemia mouse model. (Chyu K Y, et al., 2004; Goodin, M. G., et al., 2003). Non-amyloidogenic APP fragments, including α-CTF and sAPP-α, are markedly increased in the brain of transgenic APP_{sw} mice treated with EGCG versus PBS (FIG. 4A). Accordingly, soluble Aβ₁₋₄₂ and total Aβ₁₋₄₂ levels are reduced by nearly 50% and 25% respectively in EGCG treated transgenic APP_{sw} mice (FIG. 4B) by Aβ ELISAs, which are associated with a significantly elevated activity of α-secretase cleavage by 28% (FIG. 4d).

These data indicate that EGCG can act as an agonist for promotion of α-secretase cleavage of APP in this transgenic mouse model of AD. In order to test if these EGCG effects are derived from actions in the periphery and/or the central nervous system (CNS), we also administered EGCG to these mice by an intracerebroventricular (ICV) route. These EGCG-treated transgenic APP_{sw} mice show cerebral soluble Aβ levels are significantly reduced in the brain by 39% [384.65±22.49 versus 235.60±13.04 (mean pg/mg of total protein±s.e.m.)], which again are associated with increased production of α-CTF/sAPP-α (data not shown) and elevated α-secretase cleavage activity by 32% [601.8±38.13 versus 890.29±104.41 (mean fluorescence units/mg of total protein±s.e.m.)]. Importantly, cerebral soluble Aβ levels are reduced by a similar magnitude in EGCG IP-administrated transgenic APP_{sw} mice, suggesting that the in vivo effect of EGCG on anti-amyliogenic processing of APP we observed by IP route is mainly derived from actions in the CNS. Finally, we examined Aβ plaques in the brain of these mice using 4G8 immunochemistry staining analysis. At 14 months of age, 4G8 immunoreactive and thioflavin S positive Aβ deposits significantly reduced nearly 32-36% across the three brain regions examined (FIG. 4F). These data are supported by Aβ ELISA produced results that insoluble 5 M guanidine-prepared Aβ levels are decreased by approximately 26% (FIG. 4C). Together, this evidence further demonstrates that EGCG promotes non-amyloidogenic processing of APP in vivo.

It has been shown that TACE is critically involved in TNF-α maturation associated with pro-inflammatory responses (Moro, M. A. et al., 2003). However, the above examples imply that TACE may be responsible for the increased α-secretase cleavage of APP observed following EGCG treatment in SweAPP N2a cells. Interestingly, as TACE expression increases with EGCG treatment we see no associated elevation of TNF-α levels in cultured medium of microglial cells (data not shown). This phenomenon suggests that under these circumstances TACE functions predominantly in α-secretase activity rather than in TNF-α maturation and release. Additionally, reports that EGCG alone may inhibit TNF-α expression provide insight as to why we see TACE polarized in this o-secretase cleavage role (Li, R. et al., 2004; Suganuma, M. et al., 2000).

A previous study showed that EGCG might act as a β-secretase inhibitor based on a cell-free assay (Jeon, S. Y. et al., 2003). In the present application, the EGCG-reduced Aβ generation that we observed may be accomplished via inhibition of BACE activity. In order to address this question, we co-treated SweAPP N2a cells with β-secretase inhibitor II. Results show that treatment with β-secretase inhibitor II at 0.5 μM to 1.5 μM (Abbenante G. et al., 2000) failed to produce the same increased α-CTF and sAPP-α levels as EGCG as shown in FIG. 2 (data not shown). However, TAPI-1, a TACE inhibitor (Slack, B. E. et al., 2001), significantly attenuated the effect of EGCG on promoting APP α-secretase cleavage (FIGS. 3d and e). In addition, as shown in FIGS. 3b and 4d EGCG treatment markedly increases α-secretase cleavage activity in vitro and in vivo but not decreases activity of β-secretase cleavage. These data strongly suggest EGCG mainly drives α-secretase cleavage of APP in SweAPP N2a cells.

In accordance with many other green tea studies, EGCG is shown to possess great therapeutic potential. However, the observation that both GC and C can inhibit EGCG effects (FIGS. 1C and 2F) suggests that variations of these polyphenolic structures may naturally oppose or mask the beneficial properties of other flavonoids in the green tea extracts. This insight may explain why research involving green tea extracts or combinations of flavonoids results with such variable findings (Chung, F. L. et al., 2003). Furthermore, the creation of a new generation of the green tea extracts may prove to be quite prudent for therapeutic intervention in AD.

When taken together, the above data show that EGCG treatment leads to a reduction of Aβ production in both SweAPP N2a cells and transgenic APP_{sw} mouse-derived primary neuronal cells. α-CTF generation and sAPP-α secretion are markedly increased following EGCG treatment, which are correlated with elevated α-secretase cleavage activity. Furthermore, IP administration of EGCG to transgenic APP_{sw} mice results in a significantly reduced cerebral levels of Aβ concomitant with reduced Aβ plaques in the brain. In addition to IP administration, ICV injection of transgenic APP_{sw} mice also shows a reduction of cerebral Aβ levels associated with increased α-secretase cleavage activity, suggesting that EGCG effects we observed by IP route are mainly derived from actions in the CNS. Thus, EGCG actions of promoting non-amyloidogenic/α-secretase proteolytic pathway are able to reduce Aβ pathology. Because Aβ pathology in this transgenic model is representative of disease pathology in humans, EGCG administration to AD patients is expected to be an effective prophylactic strategy for reduction -of cerebral amyloidosis.

### Materials and Methods for Examples 5-7

### Reagents

Green tea-derived EGCG (95% purity by HPLC) was purchased from Sigma Chemical Co. (St Louis, Mo.). Polyclonal antibodies against ADAM10, ADAM17 (TACE), and ADAM9 were obtained from Sigma. Polyclonal antibody against the carboxyl-terminus of APP (369 antibody) was kindly provided by S. Gandy and H. Steiner. Monoclonal antibodies against the amino-terminus of APP (22C11) and against actin were purchased from Roche (Basel, Switzerland). Anti-Aβ₁₋₁₇ monoclonal antibody (6E10) and biotinylated anti-Aβ₁₇₋₂₆ monoclonal antibody (4G8) were obtained from Signet Laboratories (Dedham, Mass.).

### ELISA

Conditioned media were collected and analyzed at a 1:1 dilution using the method as previously described (Tan et al., 2002) and values were reported as percentage of Aβ₁₋ₓ secreted relative to control. Quantitation of total Aβ species was performed according to published methods (Marambaud, P. et al., 2005). Briefly, 6E10 (capture antibody) was coated at 2 μg/ml in PBS into 96-well immnunoassay plates overnight at 4° C. The plates were washed with 0.05% Tween 20 in PBS five times and blocked with blocking buffer (PBS with 1% BSA, 5% horse serum) for 2 h at room temperature. Conditioned medium or Aβ standards were added to the plates and incubated overnight at 4° C. Following 3 washes, biotinylated antibody, 4G8 (0.5 μg/mL in PBS with 1% BSA) was added to the plates and incubated for 2 h at room temperature. After 5 washes, streptavidin-horseradish peroxidase (1:200 dilutions in PBS with 1% BSA) was added to the 96-wells for 30 min at room temperature. Tetramethylbenzidine (TMB) substrate was added to the plates and incubated for 15 min at room temperature. 50 μL of stop solution (2 N N₂SO₄) was added to each well of the plates. The optical density of each well was immediately determined by a microplate reader at 450 nm. In addition, Aβ₁₋₄₀, or Aβ₁₋₄₂ was separately quantified in these samples using the Aβ₁₋₄₀ Aβ₁₋₄₂ ELISA kits (IBL-America, Minneapolis, Minn.) in accordance with the manufacturer's instructions. In all cases, Aβ levels were expressed as a percentage of control (conditioned medium from untreated SWeAPP N2a cells).

Western Blot

Cultured cells were lysed in ice-cold lysis buffer described above, and an aliquot corresponding to 50 μg of total protein was electrophoretically separated using 16.5% Tris-tricine gels. Electrophoresed proteins were then transferred to PVDF membranes (Bio-Rad, Richmond, Calif.), washed in ddH₂O, and blocked for 1 h at ambient temperature in Tris-buffered saline (TBS; Bio-Rad) containing 5% (w/v) non-fat dry milk. After blocking, membranes were hybridized for 1 h at ambient temperature with various primary antibodies. Membranes were then washed 3 times for 5 min each in ddH₂O and incubated for 1 h at ambient temperature with the appropriate HRP-conjugated secondary antibody (1:1,000, Pierce Biotechnology, Inc. Rockford, Ill.). All antibodies were diluted in TBS containing 5% (w/v) of non-fat dry milk. Blots were developed using the luminol reagent (Pierce Biotechnology). Densitometric analysis was done using the Fluor-S MultiImager™ with Quantity One™ software (Bio-Rad). For examining sAPP-α, conditioned medium was collected following treatment according to a modified protocol from Chen and Fernandez (Chen et al., 2004). sAPP-α was extracted using 3K Nanosep centrifugal filters (Pall Life Sciences, Ann Arbor, Mich.) and protein concentrate was prepared for the aforementioned electrophoresis. Antibodies used for Western blot included: antibody 369 (which recognizes the carboxyl-terminus of APP; 1:1,500), clone 22C11 (against the amino-terminus of APP; 1:1,500), clone 6E10 (against amino acids 1-17 of Aβ; 1:1,500), anti-ADAM9 (1:500), and antibodies against ADAM10 (1:500), ADAM17 (1:500) or actin (1:1,500; as an internal reference control).

In order to characterize cc-CTF detected by antibody 369 in this study, we performed an additional experiment. The blot where first hybridized with an antibody (369) specifically against the carboxyl-terminus of APP, was put in stripping solution (62.5 mM Tris-HCl, pH 6.8, 2% SDS, and 100 mM β-mercaptoethanol) and incubated at 50° C. for 30 min in a sealed plastic container in the shaking water bath. After stripping, this blot was rinsed with TBST (TBS+0.1% Tween 20) and re-blocked with TBSTM (TBST+5% non fat dry milk), and then re-probed with an antibody that recognizes Aβ₁₋₁₇ (6E10). Alternatively, membranes with identical samples were probed either with an antibody (369) or with an antibody, 6E10. As expected, the ˜11 kD band was positive for both 369 and 6E10 antibody probing, thereby confirming its identity as an α-CTF.

Primary Cultures

Breeding pairs of C57BL/6 mice were purchased from the Jackson Laboratory (Bar Harbor, Me.). Tg2576 mice were provided by the University of South Florida (NSF). Primary culture microglial cells were isolated from mouse cerebral cortices and were grown in RPMI 1640 medium supplemented with 5% fetal calf serum, 2 mM glutamine, 100 U/mL penicillin, 0.1 μg/mL streptomycin, and 0.05 mM 2-mercaptoethanol according to previously described methods (Chen et al., 2004; Chao et al., 1992). Briefly, cerebral cortices from newborn mice (1-2 day-old) were isolated under sterile conditions and were kept in 75 cm² flasks, and complete medium was added. Primary cultures were kept for 14 days so that only glial cells remained, and microglial cells were isolated by shaking flasks at 200 rpm at 37° C. in a Lab-Line incubator-shaker. More that 98% of these glial cells stained positive for MAC-1 (CD11b/CD18; Roche) confirming their identity as microglia. Mouse primary culture neuronal cells were prepared as previously described (Chen et al., 2004, Tan et al., 2000). Briefly, cerebral cortices were isolated from Tg2576 mouse embryos, between 15 and 17 days in utero, and were individually mechanically dissociated in trypsin (0.25%) individually after incubation for 15 min at 37° C. Cells were collected after centrifugation at 1,200 rpm, resuspended in DMEM supplemented with 10% fetal calf serum, 10% horse serum, uridine (33.6 μg/mL; Sigma) and fluorodeoxyuridine (13.6 μg/mL; Sigma), and seeded in 24-well collagen coated culture plates at 2.5×10⁵ cells per well. When neuronal cells were isolated from Tg2576 mice, to verify the presence of the transgene, PCR genotype analysis was performed as previously described (Tan et al., 2002) and human APP_{sw} transgene was detected in these cells (data not shown).

Small Interfering RNA Mediated Gene Silencing

SweAPP N2a cells were transfected with siRNA pre-designed to knock-down murine ADAM9, 10, or 17 mRNA (Dharmacon Inc. Lafayette, Colo.). SweAPP N2a cells were seeded in 24-well plates and cultured until they reached 70% confluence. The cells were then transfected with 50-200 nM anti-ADAM9, 10 or 17 siRNA or anti-green fluorescent protein (GFP; non-targeting control; Dharmacon) using Code-Breaker transfection reagent (Promega, Madison, Wis.) and cultured for an additional 18 h in serum-free MEM. Transfection efficiency was determined to be greater than 80% (data not shown) using no-RISC siGLOW (fluorescently labeled non-functional siRNA; Dharmacon). The cells were allowed to recover for 24 h in complete medium (MEM 10% FBS) before treatmnents. The cells were evaluated by Western blot analysis for expression of ADAM9, 10 or 17.

RT-PCR

Analysis of murine ADAM10 was conducted according to previously published methods (Ehrhart et al., 2005; Park et al., 2001). Briefly, total RNA was isolated from SweAPP N2a cells and subjected to reverse transcription utilizing a commercially available kit (cDNA Cycle® kit; Invitrogen, Carlsbad, Calif.) according to the manufacturer's instructions on a Bio-Rad iCycler thermocycler. The same machine was used to amplify murine cDNA by PCR using ADAM10 sense (5′-GCC AGC CTA TCT GTG GAA ACG GG-3′) and antisense (5′-TTA GCG TCG CAT GTG TCC CAT TTG-3′) primers or γ-actin sense (5′-TTG AGA CCT TCA ACA CCC-3′) and antisense (5′-GCA GCT CAT AGC TCT TCT-3′) primers (0.5 μg/25 μL final reaction volume) using a commercially available kit (HotStarTaq Master Mix; Qiagen, Valencia, Calif.) according to the manufacturer's instructions. Thermocycler conditions consisted of an initial denaturing step at 95° C. for 15 min, followed by 35 cycles of 94° C. for 30 s, 50° C. for 1 min, and 72° C. for 1 min at, and a final extension step at 72° C. for 10 min. Resolution and analysis of PCR products (murine ADAM10: 881 bp, murine γ-actin: 357 bp) band densities was conducted by ethidium bromide-stained agarose gel electrophoresis and identified using UV transillumination by comparisons with molecular weight markers (Invitrogen). Samples that were not subjected to reverse transcription were run in parallel as negative controls to rule out DNA contamination as a template for PCR products (data not shown). A “no template control” was also included for each primer set as a further negative control (data not shown). Amplification of γ-actin was used to normalize for input cDNA.

Statistical Analysis

All data were normally distributed; therefore, in instances of single mean comparisons, Levene's test for equality of variances followed by t-test for independent samples was used to assess significance. In instances of multiple mean comparisons, analysis of variance (ANOVA) was used, followed by post-hoc comparison using Bonferonni's method. Alpha levels were set at 0.05 for all analyses. The statistical package for the social sciences release 10.0.5 (SPSS Inc., Chicago, Ill.) was used for all data analysis.

### Example 5

### EGCG Treatment Enhances ADAM10 Activation in Cultured CNS Cells

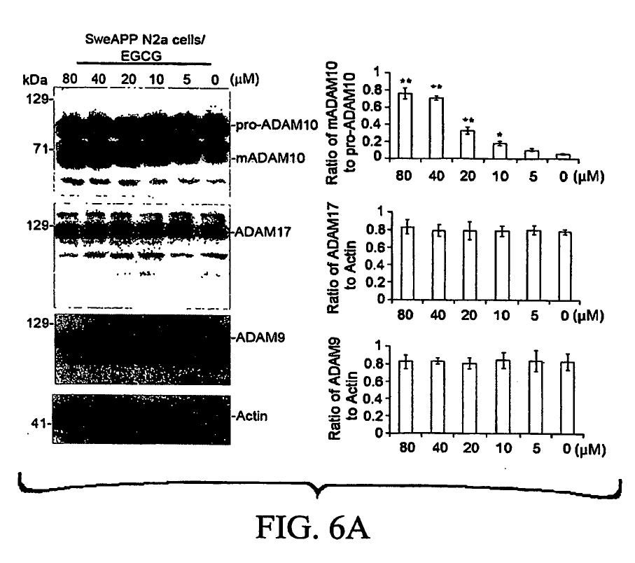

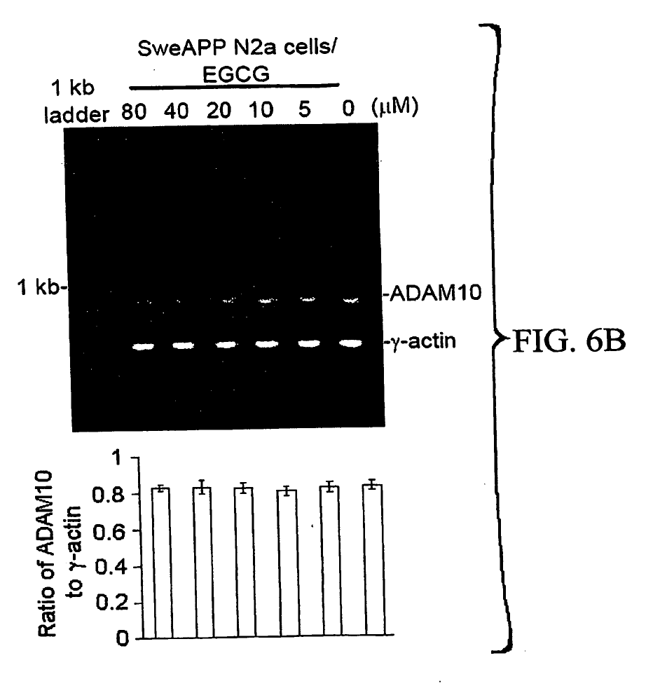

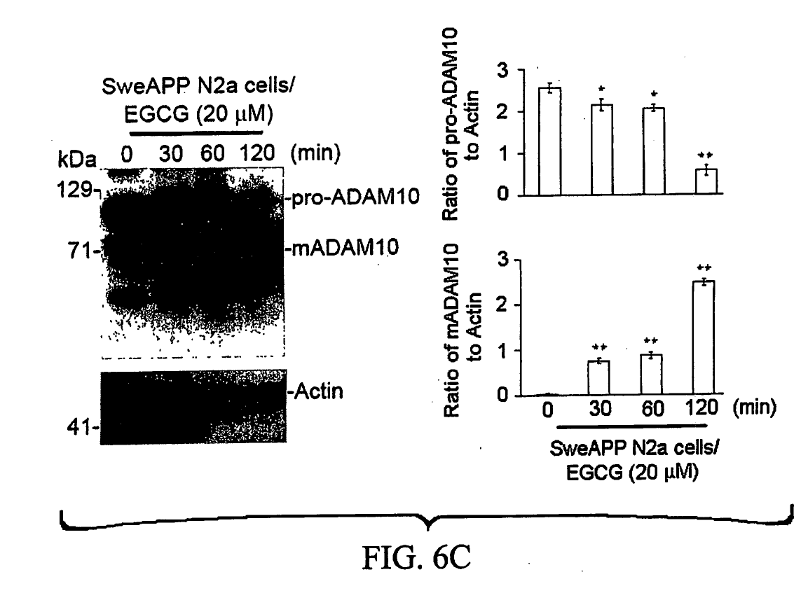

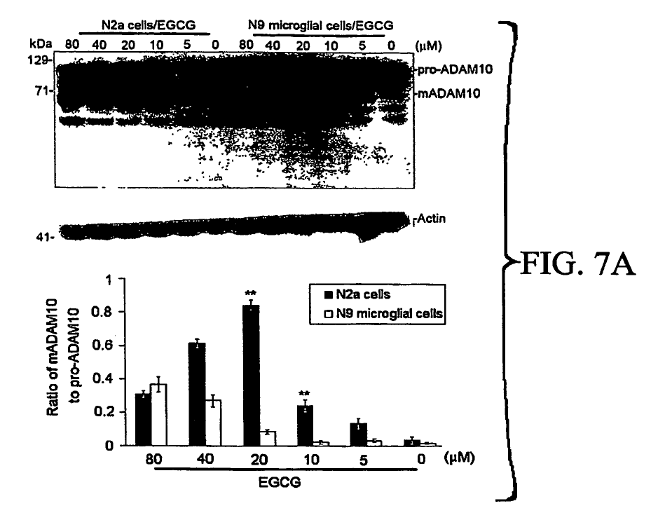

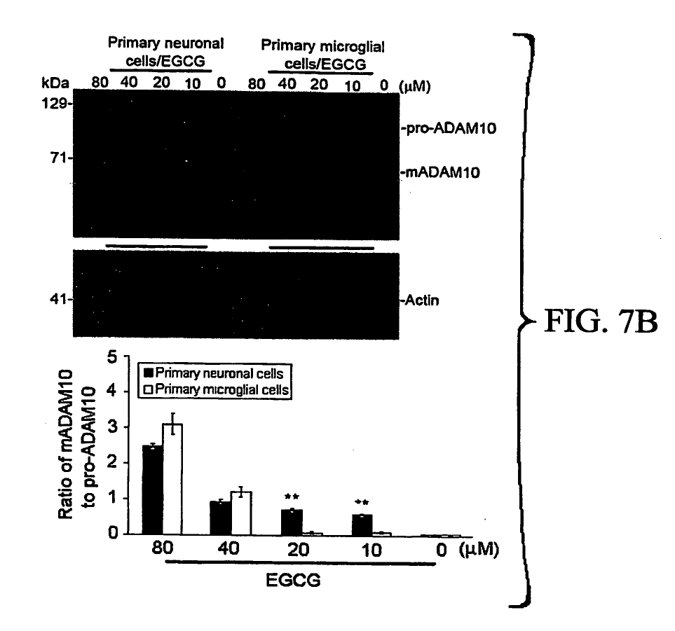

To determine whether EGCG modulates expression of candidate α-secretases ADAM9, 10 or 17, we treated N2a cells stably transfected with “Swedish” mutant APP (SweAPP N2a cells) with various doses of EGCG and measured respective levels of protein expression. Mature ADAM10 (the ˜60 kDa isoform), but not ADAM9 or ADAM17, dose-dependently increased in response to EGCG treatment (FIG. 6A). To investigate if EGCG treatment might affect mRNA expression of ADAM10 across the time-frame examined above, we isolated total RNA from cells treated in parallel for RT-PCR analysis. Results show no significant between-EGCG dose differences on ADAM10 mRNA levels (FIG. 6B). Temporal analysis of EGCG's effect on ADAMs expression showed significant increases in mature ADAM10 as early as 30 min after treatment with 20 μM of EGCG (FIG. 6C), an effect which continued to increase through to 120 min after EGCG challenge. However, no significant effects of EGCG treatment on ADAM 9 or 17 were noted (data not shown). Consistent with these findings, EGCG also dose-dependently increased ADAM10 maturation in two additional cell types, parental (non-transfected) N2a cells and N9 microglial cells. Relative to N9 microglia, the neuron-like parental N2a cell line demonstrated increased sensitivity to EGCG treatment (FIG. 7A). Similar to N2a and N9 cell lines, primary murine neuronal and microglial cultures also displayed dose-dependent increases in mature ADAM10 in response to EGCG treatment (FIG. 7B), with primary neurons showing increased sensitivity to the lower doses (10 and 20 μM) of EGCG.

### Example 6

### EGCG-Induced Maturation of ADAM10 Correlates with APP α-Secretase Cleavage

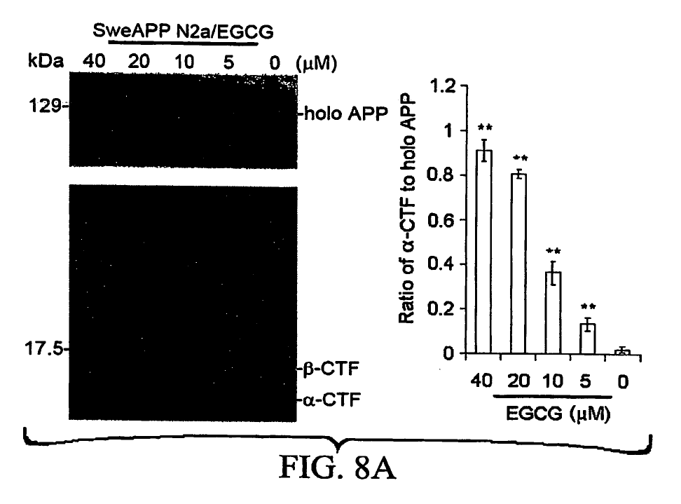

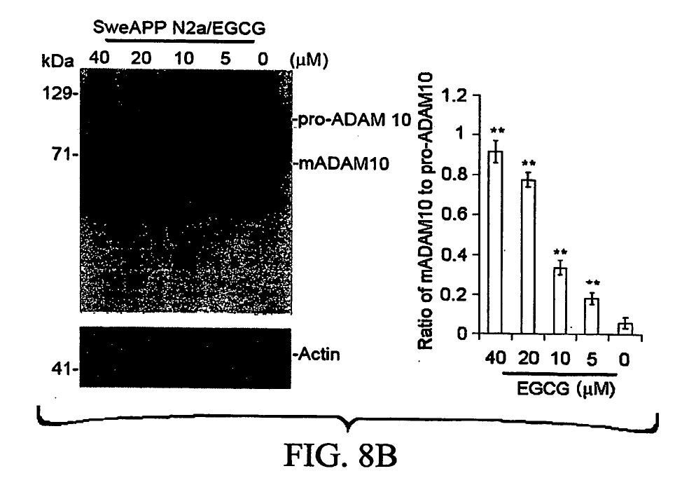

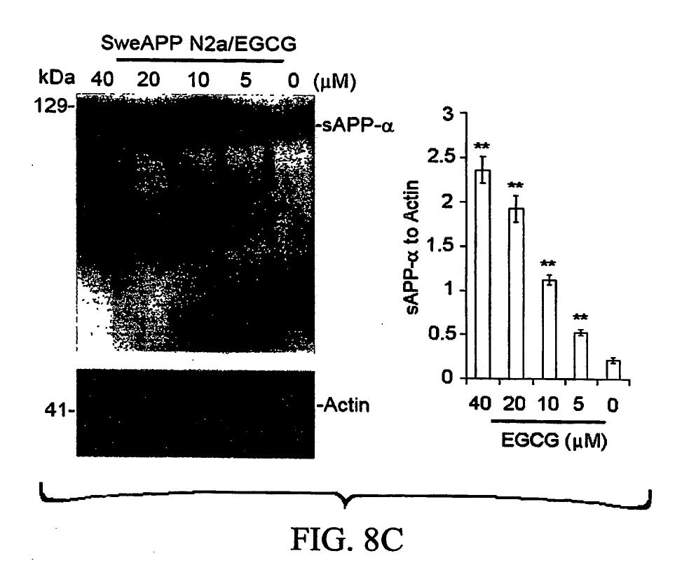

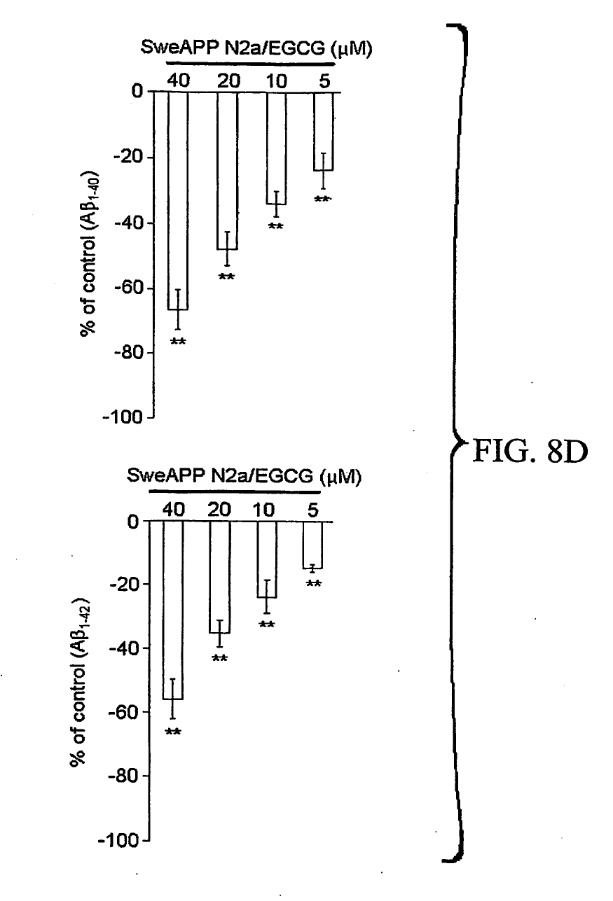

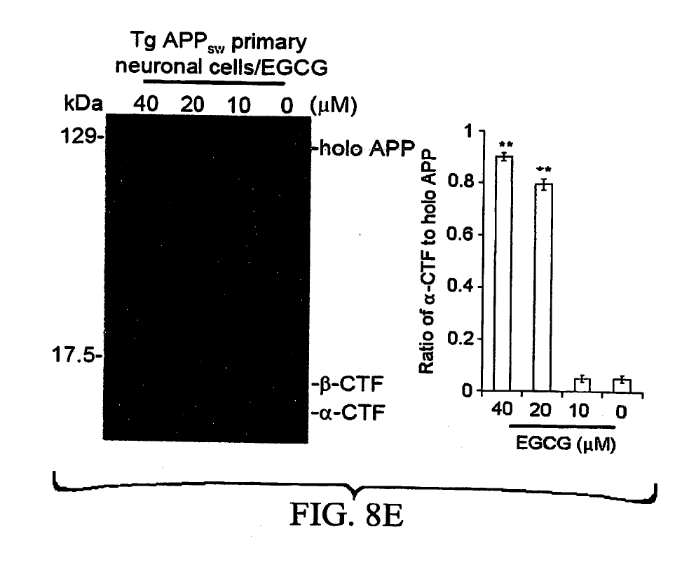

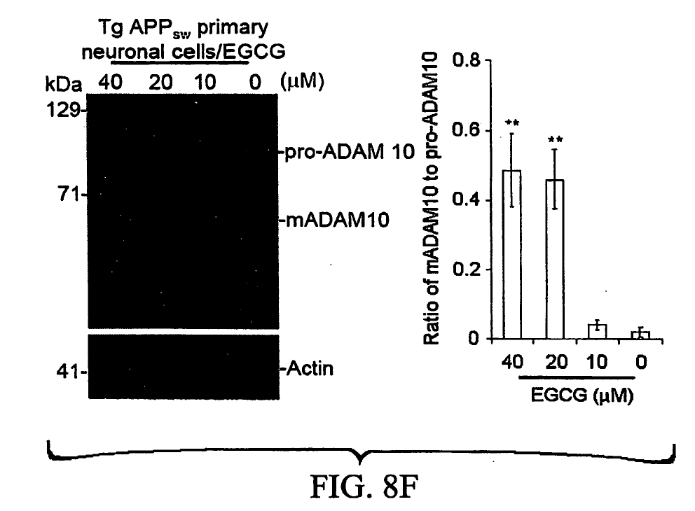

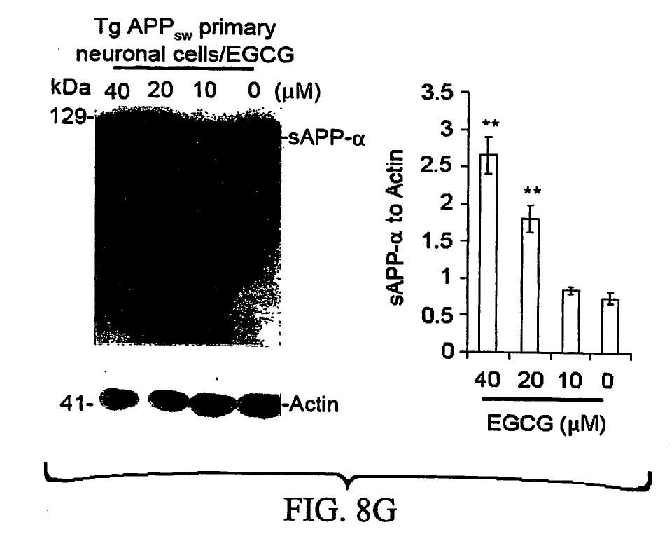

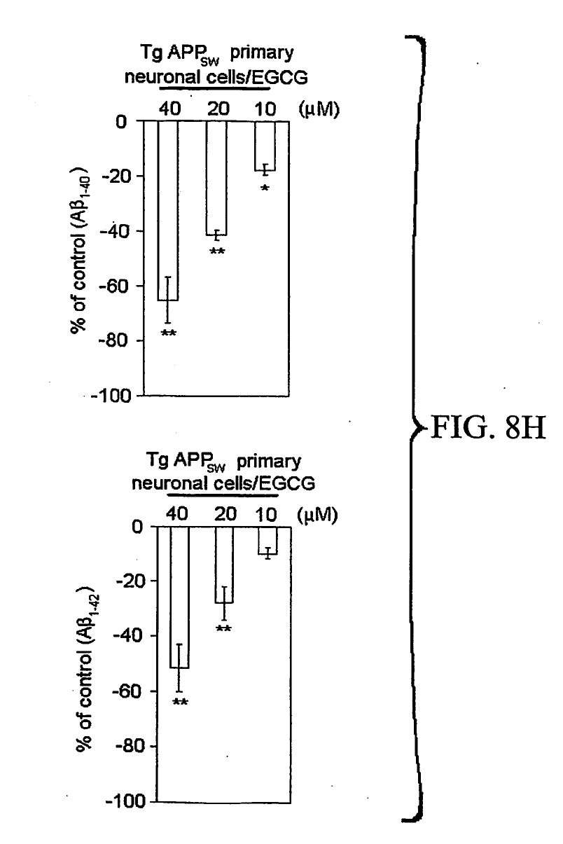

To determine whether the EGCG-mediated dose-dependent increase in mature ADAM10 might result in modulation of APP processing, we subjected SweAPP N2a cells to various doses of EGCG and then analyzed APP metabolism and ADAM10 maturation in parallel. Western analysis revealed dose-dependent increases in α-CTF and sAPP-α with corresponding increases in mature ADAM10 in response to EGCG treatment (FIGS. 8A, 8B, 8C). Moreover, we also observed EGCG dose-dependent reductions in Aβ₁₋₄₀ and Aβ₁₋₄₂ concentrations after EGCG treatment by Aβ ELISA (FIG. 8D), further connnming that 1) EGCG promotes non-amyloidogenic APP processing and 2) that this effect correlates with increased ADAM10 maturation. Accordingly, primary neuronal cells derived from Tg2576 mice were also analyzed for changes in APP metabolism in response to EGCG treatment. Western analysis revealed EGCG promotion of the APP α-secretase cleavage pathway, as quantified by the ratio of α-CTF to holo APP (FIG. 8E). Similar to data observed in SweAPP N2a cells, APP α-CTF cleavage positively correlated with mature ADAM10 levels (FIG. 8F) and with secreted sAPP-α (FIG. 8G) in these cells. Importantly, we also observed dose-dependent reductions in Aβ₁₋₄₀ and Aβ₁₋₄₂ levels following EGCG treatment of primary neurons from Tg2576 mice (FIG. 8H).

### Example 7

### ADAM10 is Required for EGCG-Induced APP α-Secretase Cleavage

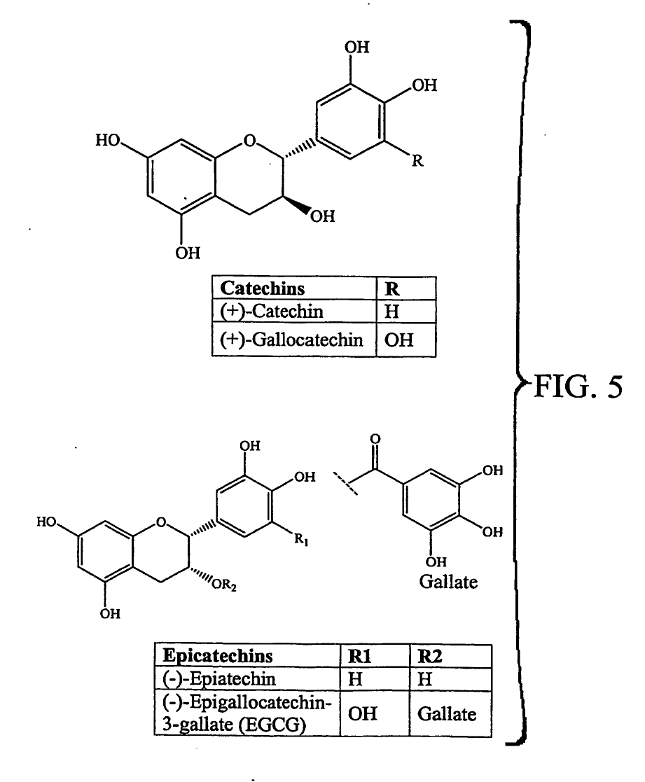

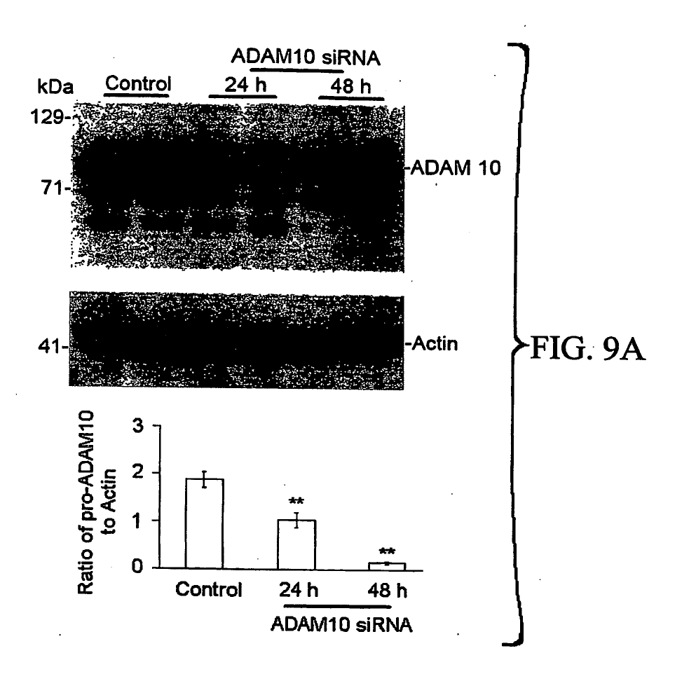

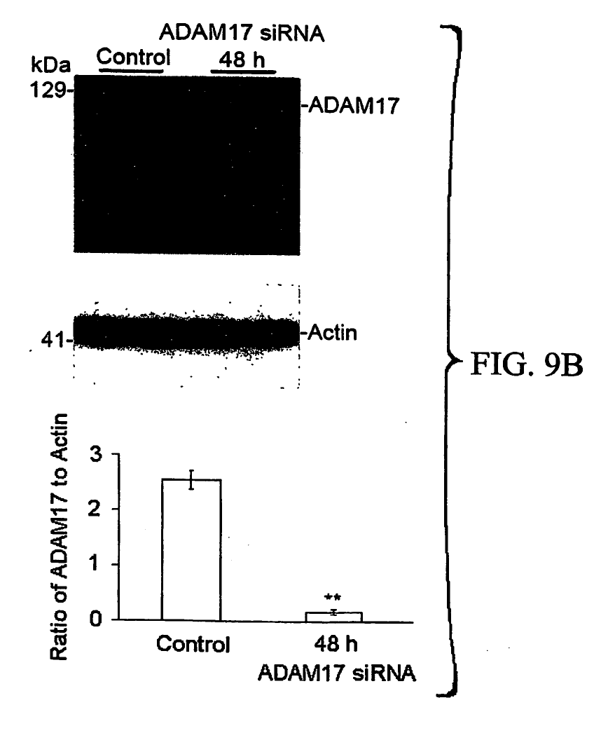

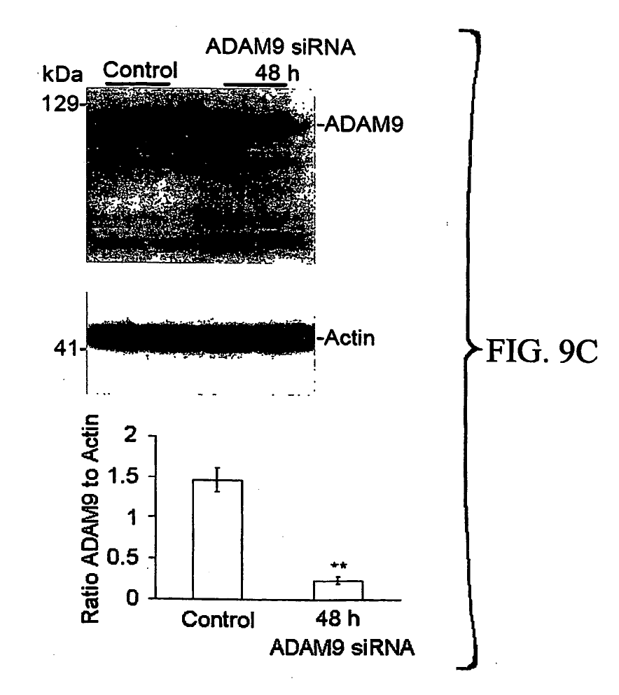

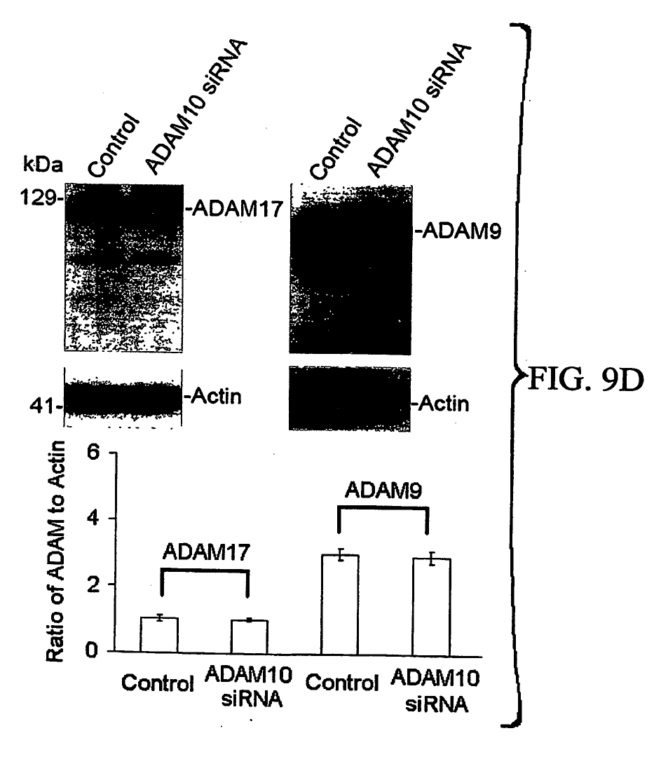

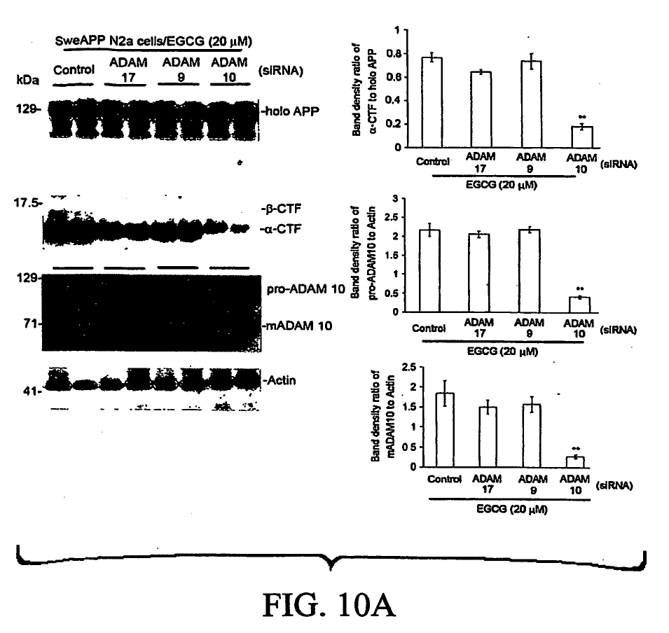

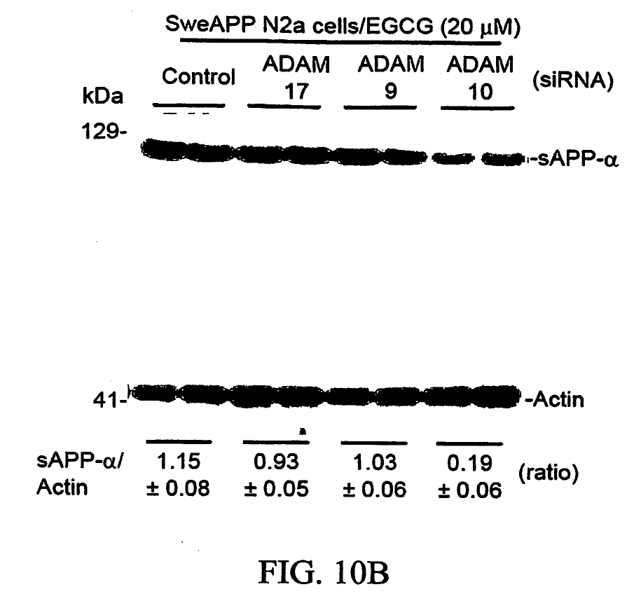

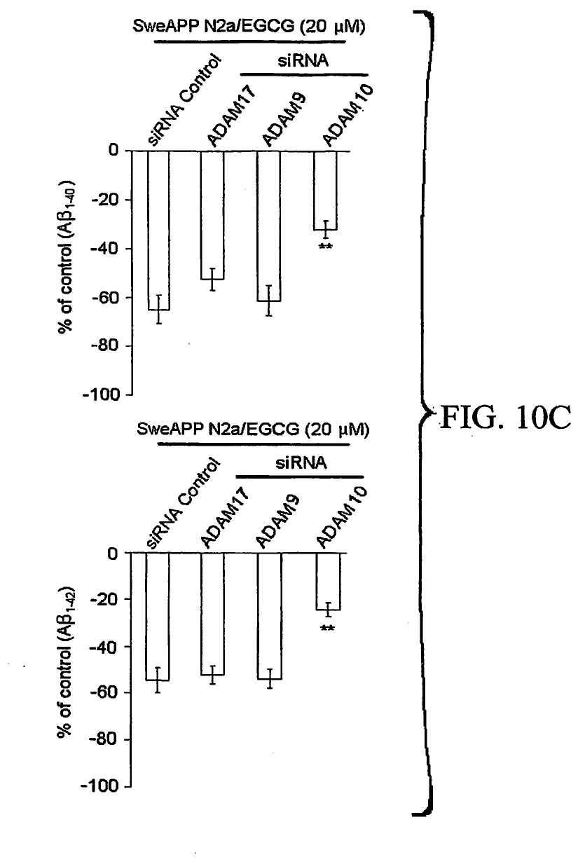

To directly examine whether ADAM o-secretase activity was required for EGCG promotion of non-amyloidogenic APP cleavage, we conducted siRNA knock-down experiments targeting ADAM9, 10 or 17. First, to confirm siRNA knock-down efficiency, SweAPP N2a cells were treated with ADAM9, 10, or 17 siRNAs and then Western blotted for expression of respective ADAMs. As shown in FIGS. 9A, 9B, and 9C, protein expression levels of ADAM10, 17, or 9 were significantly inhibited by respective ADAM-specific siRNAs. In addition, to test the specificity of siRNA against ADAM10 versus ADAM9 or 17, we analyzed expression of ADAM9 and 17 in cell lysates derived from siRNA knock-down cells for ADAM10 using Western blot. Results show that ADAM10 siRNA does not alter the expression of ADAM9 and 17 (FIG. 9D). We next examined α-CTF production in SweAPP N2a cells subjected to siRNA knock-down of ADAMs following treatment with 20 μM of EGCG, and observed that only ADAM10 siRNA was able to clearly 1) inhibit expression of ADAM10 as evidenced by decreased band density ratios of pro-ADAM10 to actin and MADAM10 to actin, and 2) block EGCG-induced α-CTF production and sAPP-α secretion (FIGS. 10A and 10B). This effect of ADAM10 siRNA on blocking EGCG-induced non-amyloidogenic APP processing was further borne out by Aβ ELISA analysis, where only ADAM10 siRNA attenuated EGCG-induced reduction of Aβ₁₋₄₀ and Aβ₁₋₄₂ (FIG. 5c). Taken together, these data demonstrate the requirement of ADAM10 for EGCG-mediated promotion of APP α-secretase cleavage.

Any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

Abbenante, G. et al., (2000) “Inhibitors of beta-amyloid formation based on the beta-secretase cleavage site” Biochem Biophys Res Commun., 268:133-5.
Ahmed S. et al., (2002) “Green tea polyphenol epigallocatechin-3-gallate inhibits the IL-1 beta-induced activity and expression of cyclooxygenase-2 and nitric oxide synthase-2 in human chondrocytes” Free Radic Bio Med 33:1097-1105.
Aktas O. et al., (2004) “Green tea epigallocatechin-3-gallate mediates T cellular NF-kappaB inhibition and exerts neuroprotection in autoimmune encephalomyelitis” J. Immunol 173:5794-5800.
Allinson, T. M. et al., (2003) J. Neurosci. Res. 74:342-352.
Asai, M. et al., (2003) Biochem. Biophys. Res. Commun. 301:231-235.
Bernstein, H. G. et al., (2003) J. Neurocytol. 32:153-160.
Buxbaum, J. D. et al., (1998) J. Biol. Chem. 273:27765-27767.
Chao, C. C., et al., (1992) J. Infect. Dis. 166:847-853
Chauhan N. B. and Siegel G. J. (2003) “Intracerebroventricular passive immunization with anti-Abeta antibody in Tg2576” J. Neurosci Res 74:142-147.
Chung, F. L. et al., (2003) “Tea and cancer prevention: studies in animals and humans” J Nutr., 133:3268S-3274S.
Chen, M., and Fernandez, H. L. (2004) Biochem. Biophys. Res. Commun. 316:332-340.
Chyu, K. Y. et al., (2004) “Differential effects of green tea-derived catechin on developing versus established atherosclerosis in apolipoprotein E-null mice” Circulation, 109:2448-53.
Citron et al. (1994) Proc. Natl. Acad. Sci. U.S.A. 91:11993-11997.
Colciaghi, F. et al., (2002) Mol. Med. 8:67-74.
Colciaghi, F. et al., (2004) Neurology 62:498-501.
De Strooper B. et al., (1998) “Deficiency of presenilin-1 inhibits the normal cleavage of amyloid precursor protein” Nature 391:387-390.
Ehrhart, J et al., (2005) J. Neuroinflammation 2:29.
Funamoto S. et al., (2004) “Truncated carboxyl-terminal fragments of beta-amyloid precursor protein are processed to amyloid beta-proteins 40 and 42” Biochemistry 43:13532-13540.
Gandhi S. et al., (2004) “Amyloid precursor protein compartmentalization restricts beta-amyloid production: therapeutic targets based on BACE compartmentalization” J. Mol Neurosci 24:137-143.
Goodin, M. G. et al., (2003) “Epigallocatechin gallate modulates CYP450 isoforms in the female Swiss-Webster mouse” Toxicol Sci., 76:262-70.
Golde T. E. et al., (2000) “Biochemical detection of Abeta isoforms: implications for pathogenesis, diagnosis, and treatment of Alzheimer's disease” Biochim Biophys Acta 1502:172-187.
Han M. K. (2003) “Epigallocatechin gallate, a constituent of green tea, suppresses cytokine-induced pancreatic beta-cell damage” Exp Mol Med 35:136-139.
Hardy J. and Selkoe D. J. (2002) “The amyloid hypothesis of Alzheimer's disease: progress and problems on the road to therapeutics” Science 297:353-356.
Hooper N. M. and Turner A. J. (2002) “The search for alpha-secretase and its potential as a therapeutic approach to Alzheimer's disease” Curr Med Chem 9:1107-1119.
Hotoda, N. et al., (2002) Biochem. Biophys. Res. Commun. 293:800-805.
Hsiao K, et al., (1996) “Correlative memory, deficits, Abeta elevation, and amnyloid plaques in transgenic mice” Science 274:99-102.
Huse J. T. et al., (2000) “Closing in on the amyloid cascade: recent insights into the cell biology of Alzheimer's disease” Mol Neurobiol 22:81-98.
Jeon S. Y. et al., (2003) “Green tea catechins as a BACE1 (beta-secretase) inhibitor” Bioorg Med Chem Lett., 13: 3905-8.
Lam, W. H. et al., (2004) “A Potential Prodrug for a Green Tea Polyphenol Proteasome Inhibitor: Evaluation of the Peracetate Ester of (−)-Epigallocatechin Gallate [(−)-EGCG]” Bioorganic & Medicinal Chemistry, 12:5587-5593.
Lammich, S. et al. (1999) Proc. Natl. Acad. Sci. U.S.A. 96:3922-3927.
Li et al., (2001) “Enantioselective Synthesis of Epigallocatechin-3-gallate (EGCG), the Active Polyphenol Component from Green Tea” Organic Letters, 3:739-41.
Li, R. et al., (2004) “(−)-Epigallocatechin gallate inhibits lipopolysaccharide-induced microglial activation and protects against inflammation-mediated dopaminergic neuronal injury” J Neurosci Res., 78:723-31.
Lichtenthaler et al., (2004) “Amyloid at the cutting edge: activation of α-secretase prevents amyloidogenesis in an Alzheimer disease mouse model” J. Clin. Invest., 113:1384-87.
Lin J. K. and Liang Y. C. (2000) “Cancer chemoprevention by tea polyphenols” Proc Natl Sci Counc Repub China 1:1-13.
Levites Y. et al., (2002) “Involvement of protein kinase C activation and cell survival/cell cycle genes in green tea polyphenol (−)-epigallocatechin 3-gallate neuroprotective action” J Biol Chem 277:30574-30580.
Levites Y. et al., (2003) “Neuroprotection and neurorescue against Abeta toxicity and PKC-dependent release of nonamyloidogenic soluble precursor protein by green tea polyphenol (−)-epigallocatechin-3-gallate” FASEB J 17:952-954.
Lopez-Perez, E. et al., (2001) J. Neurochem. 76:1532-1539.
Marambaud, P. et al., (2005) J. Biol. Chem. 45:37377-37382.
Moro M. A. et al., (2003) “Expression and function of tumour necrosis factor-alpha-converting enzyme in the central nervous system” Neurosignals 12:53-58.
Moyers S. B. and Kumar N. B. (2004) “Green tea polyphenols and cancer chemoprevention: multiple mechanisms and endpoints for phase II trials” Nutr Rev 62:204-211.
Mullan, M. et al. (1992) “A pathogenic mutation for probable Alzheimer's disease in the APP gene at the N-terminus of beta-amyloid” Nat. Genet. 1:345-347.
Okello et al., (2004) “In vitro Anti-β-secretase and Duel Anti-cholinesterase Activities of Camellia sinensis L. (tea) Relevant to Treatment of Dementia” Phytother. Res., 18: 624-27.
Park, J. W. et al., (2001) Biochem. Biophys. Res. Commun. 286:721-725.
Postina, R. et al., (2004) J. Clin. Invest 113:1456-1464.
Remington's Pharmaceutical Science (Martin E W (1995) Easton Pa., Mack Publishing Company, 19^{th} ed.
Rezai-Zadeh, K. et al. (2005) “Green tea epigallocatechin-3-gallate (EGCG) modulates amyloid precursor proten cleavage and reduces cerebral amyloidosis in Alzheimer transgenic mice” J. Neural Sci. 25(38):8807-8814.
Rossner S. et al., (2000) “Constitutive overactivation of protein kinase C in guinea pig brain increases alpha-secretory APP processing without decreasing beta-amyloid generation” Eur J Neurosci 12:3191-3200.
Sambamurti K. et al., (2002) “Advances in the cellular and molecular biology of the beta-amyloid protein in Alzheimer's disease” Neuromolecular Med 1:1-31.
Sinha S. and Lieberburg I. (1999) “Cellular mechanisms of beta-amyloid production and secretion” Proc Natl Acad Sci USA 96:11049-11053.
Slack, B. E. et al., (2001) “Constitutive shedding of the amyloid precursor protein ectodomain is up-regulated by tumour necrosis factor-alpha converting enzyme” Biochem J., 357:787-94.
Smith, D. M. et al., (2004) “Docking Studies and Model Development of Tea Polyphenol Proteasome Inhibitors: Applications to Rational Drug Design” PROTEINS: Structure, Function, and Bioimformatics, 54:58-70.
Steiner H. et al., (1999) “A loss of function mutation of presenilin-2 interferes with amyloid beta-peptide production and notch signaling” J. Biol Chem 274:28669-28673.
Suganuma, M. et al., (2000) “Mechanisms of cancer prevention by tea polyphenols based on inhibition of TNF-alpha expression” Biofactors, 13:67-72.
Tan et al., (2000) J. Neurosci. 20:7587-7594.
Tan et al., (2002) “Role of CD40 ligand in amyloidosis in transgenic Alzheimer's mice” Nat Neurosci, 5:1288-1293.
Wan, S. B. et al., (2005) “Structure-activity Study of Epi-gallocatechin Gallate (EGCG) Analogs as Proteasome Inhibitors” Bioorganic & Medicinal Chemistry, 13:2177-2185.
Yan R. et al., (1999) “Membrane-anchored aspartyl protease with Alzheimer's disease beta-secretase activity” Nature 402:533-537.

1-85. (canceled)

86. A method for:
i) treating or preventing a neurodegenerative disease or condition associated with β-amyloid peptide deposition in neural tissue, said method comprising administering to a person or animal in need thereof an effective amount of a polyphenol, or an analog, isomer, metabolite, or prodrug thereof, that increases expression or activity of a protein having α-secretase enzymatic activity; or
ii) decreasing or inhibiting deposition of β-amyloid peptide in neural tissue, said method comprising contacting said neural tissue with an effective amount of a polyphenol, or an analog, isomer, metabolite, or prodrug thereof, that increases expression or activity of a protein having α-secretase enzymatic activity; or
iii) decreasing levels of a β-amyloid peptide produced by a cell, said method comprising contacting a cell with an effective amount of a polyphenol, or an analog, isomer, metabolite, or prodrug thereof, that increases expression or activity of a protein having α-secretase enzymatic activity.

87. The method according to claim 86, wherein said polyphenol is epigallocatechin-3-gallate (EGCG) and epicatechin (EC).

88. The method according to claim 86, wherein said neurodegenerative disease or condition is Alzheimer's disease.

89. The method according to claim 86, wherein said neural tissue is central nervous system tissue.

90. The method according to claim 86, wherein said neural tissue is brain tissue.

91. The method according to claim 86, wherein said polyphenol is purified to a level that compounds that antagonize expression or activity of said protein are not present in detectable amounts.

92. The method according to claim 91, wherein said compounds that antagonize α-secretase activity are gallocatechin and (−)catechin.

93. The method according to claim 86, wherein said polyphenol is provided in a pharmaceutically acceptable composition.

94. The method according to claim 93, wherein said pharmaceutically acceptable composition comprises pharmaceutically acceptable salts of said polyphenol.

95. The method according to claim 86, wherein said polyphenol is administered orally, rectally, nasally, topically, vaginally, parenterally, or by pulmonary administration, or by intra-arterial, intramuscular, intravenous, or intraperitoneal injection.

96. The method according to claim 93, wherein said pharmaceutical composition is formulated as an ointment, cream, suspension, lotion, powder, solution, paste, gel, spray, aerosol, or oil.

97. The method according to claim 86, wherein said polyphenol is provided in a purified form.

98. The method according to claim 86, wherein said polyphenol is administered simultaneously or sequentially with a compound or agent selected from the group consisting of galantamine, deprenyl, cdp choline, folate, Vitamin B12, Vitamin B6, piracetam, vinpocetine, idebenone, pyritinol, memantine, anti-oxidants, free radical scavenging agents, peptides, growth factors, antibiotics, bacteriostatic agents, immunosupressives, anti-coagulants, buffering agents, anti-inflammatory agents, anti-pyretics, time released binders, anesthetics, steroids, corticosteroids, or a combination of any of the forgoing.

99. The method according to claim 86, wherein said method further comprises administering compounds or agents that inhibit or decrease expression or activity of a protein having β-secretase activity or γ-secretase activity.

100. The method according to claim, 86 wherein the method further comprises identifying a person or animal afflicted with or at risk of developing said neurodegenerative disease or condition.

101. The method according to claim 86, wherein said α-secretase enzyme is ADAM10.

102. The method according to claim 86, wherein said cell is a neural cell.

103. A method for inducing or promoting a neurodegenerative disease or condition associated with β-amyloid deposition in an animal, said method comprising administering to an animal an effective amount of an agent or compound that inhibits expression or activity of a protein having α-secretase enzymatic activity.

104. A method for screening for candidate drugs or compounds that can be used to treat or prevent a neurodegenerative disease or condition in a person or animal, said method comprising assaying said drug or compound to determine if said drug or compound increases expression or activity of an α-secretase enzyme.

105. A method for increasing production of α-CTF and/or s-APP-α by a cell, said method comprising contacting said cell with an effective amount of a polyphenol, or an analog, isomer, metabolite, or prodrug thereof, that increases expression or activity of a protein having α-secretase enzymatic activity.

106. A composition comprising a polyphenol, or an analog, isomer, metabolite, or prodrug thereof, that increases expression or activity of a protein having α-secretase enzymatic activity, and, optionally, an agent or compound that inhibits or decreases expression or levels of protein having β-secretase enzymatic activity or γ-secretase enzymatic activity, and, optionally, a pharmaceutically acceptable carrier or diluent.

107. The composition according to claim 106, wherein said polyphenol is EGCG or EC.

108. The composition according to claim 106, wherein said polyphenol is provided in purified form.

109. The composition according to claim 108, wherein said polyphenol is purified to a level wherein compounds that antagonize the activity of the polyphenols are removed or decreased to a level wherein they do not antagonize the action of said polyphenol.

110. The composition according to claim 106, wherein said agent or compound comprises nucleic acid that is antisense to nucleic acid encoding a protein with β-secretase activity or γ-secretase activity, or said agent or compound comprises a small interfering RNA molecule that interferes with expression of a protein having β-secretase activity or γ-secretase activity.
